(19) United States
(12) Patent Application Publication
(10) Pub. No.: US 2024/0050408 A1
(43) Pub. Date: Feb. 15, 2024

(54) USE OF MGLUR5 ANTAGONISTS FOR TREATING AMPHETAMINE ADDICTION

(71) Applicant: NOVARTIS AG, Basel (CH)

(72) Inventors: Fabrizio GASPARINI, Basel (CH); Baltazar GOMEZ-MANCILLA,, Basel (CH)

(73) Assignee: NOVARTIS AG, Basel (CH)

(21) Appl. No.: 18/266,427

(22) PCT Filed: Dec. 9, 2021
(86) PCT No.: PCT/IB2021/061503

§ 371 Date: Jun. 9, 2023

Related U.S. Application Data
(60) Provisional application No. 63/124,565, filed on Dec. 11, 2020.

(51) Int. Cl. A61K 31/404; A61K 31/55; A61K 31/137; A61K 31/551; A61P 25/30

(52) U.S. Cl. CPC A61K 31/404; A61K 31/55; A61K 31/137; A61K 31/551; A61P 25/30

(57) ABSTRACT

The invention relates to the mavoglurant, or a pharmaceutically acceptable salt thereof: in the treatment of substance use disorder, wherein the substance is an amphetamine-type stimulant; in a treatment in the reduction of substance use by a substance use disorder patient, wherein the substance is an amphetamine-type stimulant; in a treatment to prevent relapse into substance use by a substance use disorder patient, wherein the substance is an amphetamine-type stimulant; in a treatment to promote substance abstinence by a substance use disorder patient, wherein the substance is an amphetamine-type stimulant; in the treatment of the symptoms of depression or anxiety associated with substance use disorder, wherein the substance is an amphetamine-type stimulant.

The present invention relates to the use of mavoglurant in the treatment of substance use disorder, wherein the substance is an amphetamine-type stimulant.

### FIELD OF THE INVENTION

The invention relates to the use of the mGluR5 antagonist named mavoglurant, or a pharmaceutically acceptable salt thereof, in the treatment of substance use disorder, wherein the substance is an amphetamine-type stimulant; in a treatment in the reduction of substance use by a substance use disorder patient, wherein the substance is an amphetamine-type stimulant; in a treatment to prevent relapse into substance use by a substance use disorder patient, wherein the substance is an amphetamine-type stimulant; in a treatment to promote substance abstinence by a substance use disorder patient, wherein the substance is an amphetamine-type stimulant; in the treatment of the symptoms of depression or anxiety associated with substance use disorder, wherein the substance is an amphetamine-type stimulant.

### BACKGROUND OF THE INVENTION

The mGlu5 receptor (mGluR5) has been implicated in the rewarding effect of various drugs of abuse and drug-seeking behaviour. In a rat model, Gass et al. Neuropsychopharm 34 (2009) 820-830 showed that the mGluR5 antagonist 3-[(2-methyl-1,3-thiazol-4-yl)ethynyl]pyridine (MTEP) dose dependently reduced the methamphetamine self-administration behavior without altering overall responding for food. MTEP also dose dependently prevented cue- and drug-induced reinstatement of methamphetamine-seeking behavior, but did not alter cue-induced reinstatement of food-seeking behavior. Herrold et al. Eur Neuropsychopharm 23 (2013) 691-696 showed that administration of either 2-Methyl-6-(phenylethynyl)-pyridine (MPEP) or 3-[(2-methyl-1,3-thiazol-4-yl)ethynyl]pyridine (MTEP) inhibited the maintenance of the expression of methamphetamine conditioned place preference in rats.

Substance use disorder, wherein the substance is an amphetamine-type stimulant, is a complex psychiatric disorder (e.g. defined with reference to DSM-5 criteria; i.e. according to the Diagnostic and Statistical Manual of Mental Disorders. 5^{th} Edition, Washington, DC: American Psychiatric Association, 2013), which continues to grow into a significant worldwide health problem having adverse medical, social and economic effects (Ling et al Current Psychiatry, Vol. 13, No.9, 37-44).

To date, there is no medication approved by the US Food and Drug Administration (FDA) for use in the treatment of this disorder. Accordingly, there is a need to identify therapeutic agents that can be used to treat it, in particular drugs that are effective on promoting abstinence and reducing relapse once patients are abstinent.

### SUMMARY OF THE INVENTION

The invention relates to the use of mavoglurant, or a pharmaceutically acceptable salt thereof:
in the treatment of substance use disorder, wherein the substance is an amphetamine-type stimulant;
in a treatment in the reduction of substance use by a substance use disorder patient, wherein the substance is an amphetamine-type stimulant;
in a treatment to prevent relapse into substance use by a substance use disorder patient, wherein the substance is an amphetamine-type stimulant;
in a treatment to promote substance abstinence by a substance use disorder patient, wherein the substance is an amphetamine-type stimulant;
in the treatment of the symptoms of depression or anxiety associated with substance use disorder, wherein the substance is an amphetamine-type stimulant.

### BRIEF DESCRIPTION OF DRAWINGS

FIG. 1A. Study Design.

FIG. 1B. Screening, Randomization and Treatment [n=number of subjects].

FIG. 2A. Cocaine Use Days: Mean (SE) of the proportion of cocaine use days. * Mavoglurant 50 mg bid from Day 1 to Day 7 followed by 100 mg bid from Day 8 to Day 14, and then fixed-dose 200 mg bid for 84 days. N=number of subjects.

FIG. 2B. Cocaine Use Days: Posterior distribution of the Bayesian analysis of the proportion of cocaine use days over the treatment period [n=number of subjects]. Probabilities were obtained using a Bayesian model including treatment as a factor and past consumption of cocaine as a covariate. The prior distributions were assumed to be uninformative. Past consumption of cocaine is the proportion of cocaine use days over the 28 days preceding the screening visit, assessed retrospectively using the TFLB. The shaded area represents a difference between mavoglurant and placebo ≥-0.1 with 36.6% confidence. The dotted line represents the observed difference between mavoglurant and placebo (−0.087); P (θ_{mavoglurant}−θ_{placebo}≥0|data)=0.999 [PoC criterion 1 is met (p>0.90)]; P(θ_{mavoglurant}−θ_{placebo}≥0% |data)=0.366 [PoC criterion 2 is not met (p<0.50)].

FIG. 2C. Cocaine Use Days: Overall proportion of cocaine use days over the treatment period.

#ANCOVA model with treatment as a factor and baseline cocaine use as covariates. † Linear mixed model for repeated measures with treatment, time, and treatment by time interaction as fixed effects and baseline cocaine use as a covariate. An unstructured covariance matrix was used. The baseline proportion is calculated over the 28 days before the screening visit.

CI=confidence interval; SE=standard error.

FIG. 3. Urine Measurements of Benzoylecgonine. Mean (SE) of log-transformed urine benzoylecgonine (BE) by week and treatment. BE=benzoylecgonine, N=total number of subjects in the intervention group.

FIG. 4. Cumulative proportion of patients abstinent from alcohol by treatment and number of weeks. N=total number of subjects in the intervention group, n=number of abstinent subjects at prespecified time point, m=number of subjects evaluable. A subject absent at the visit is non abstinent.

FIG. 5. Adverse events. N=total number of subjects in the intervention group, n=number of subjects with adverse events. AEs=adverse events.

FIG. 6. Effect of mavoglurant on alcohol use reduction: Mean (SE) of the proportion of alcohol use days. N=number of subjects.

FIG. 7. Effect of mavoglurant on alcohol use reduction: Overall proportion of alcohol use days over the treatment period. The baseline proportion is calculated over the 28 days before the screening visit.

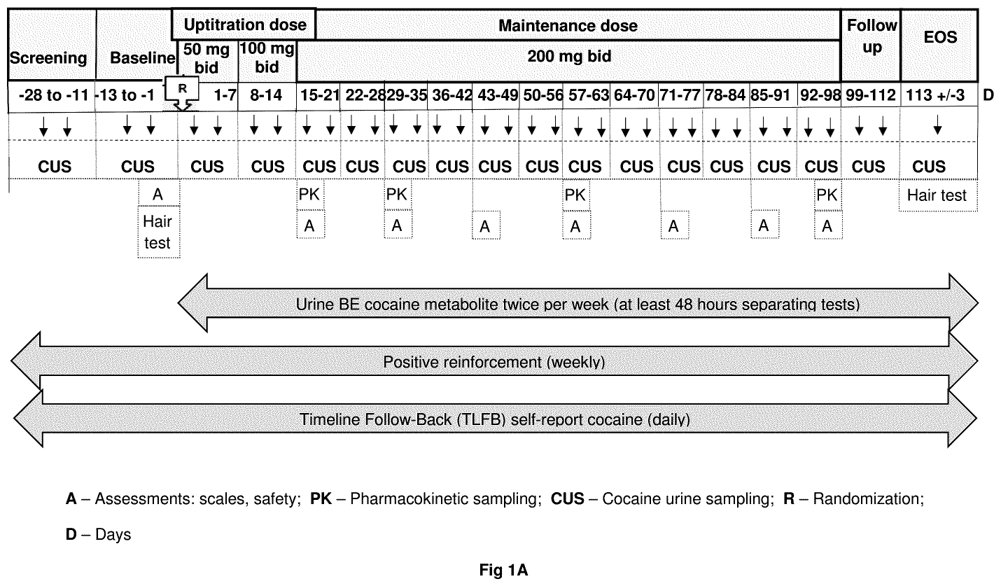

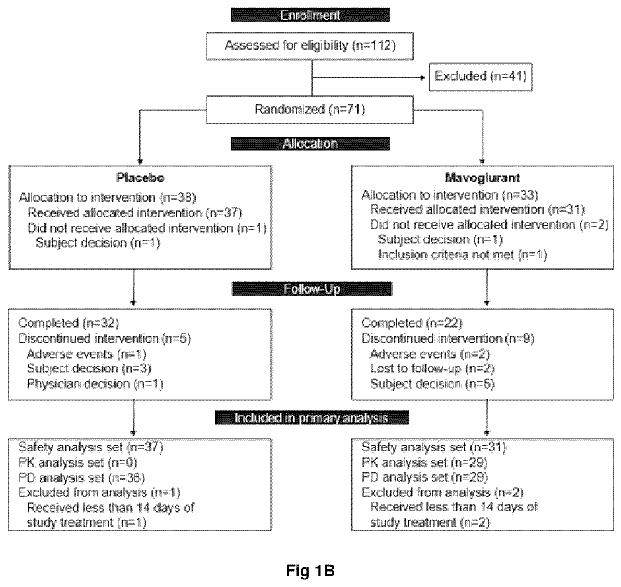

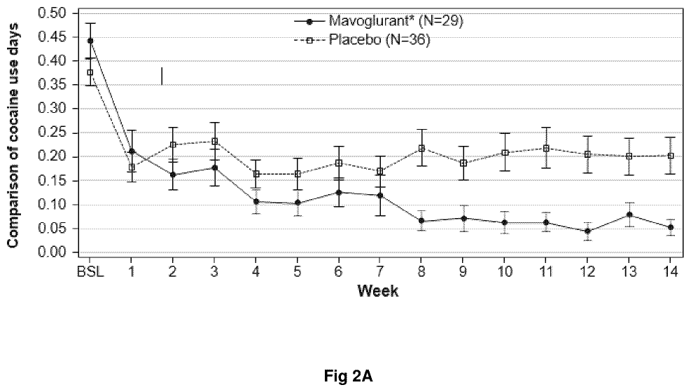

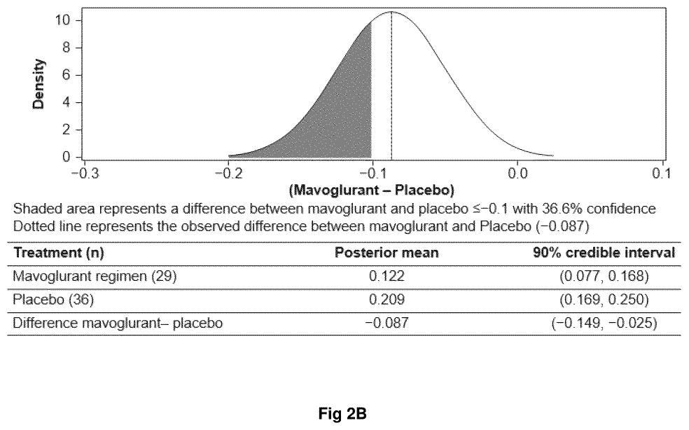

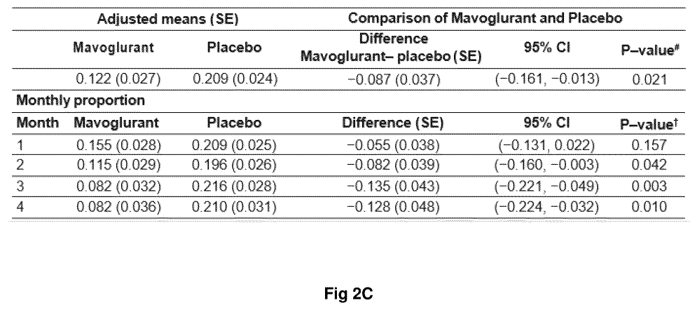

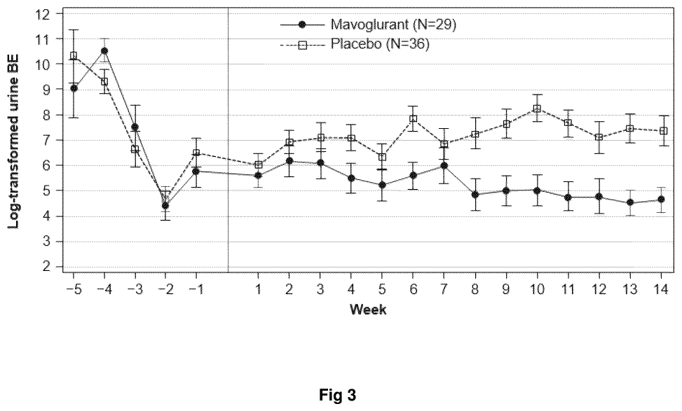

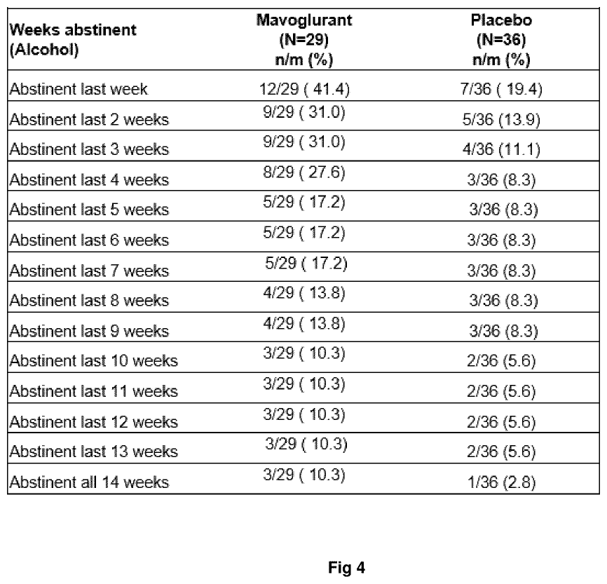

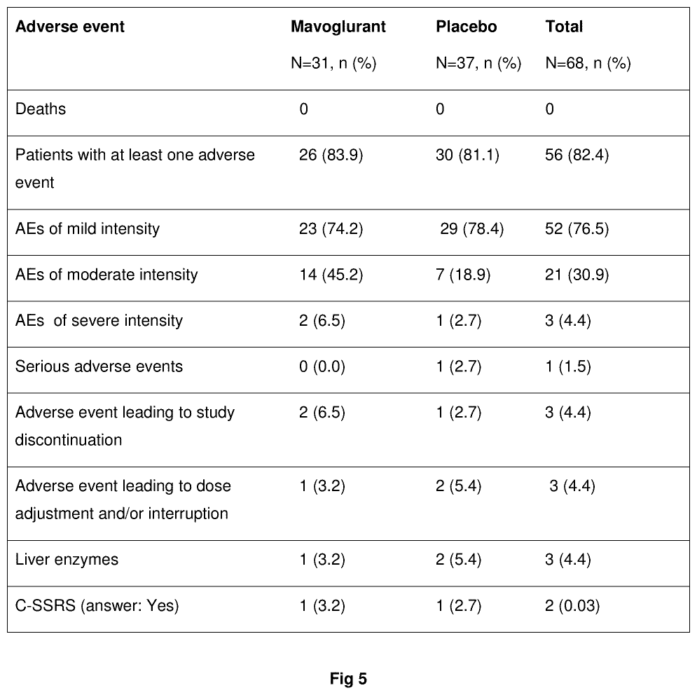

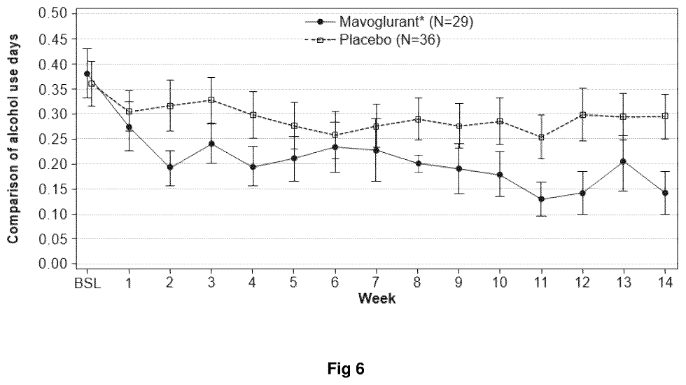

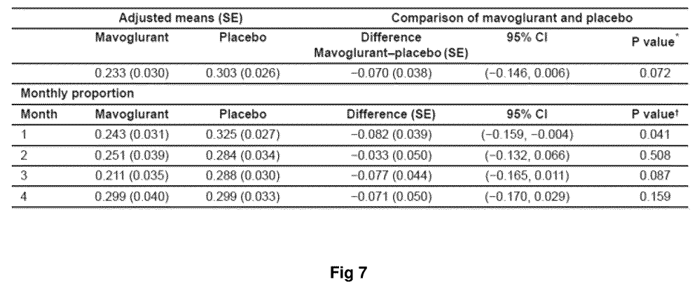

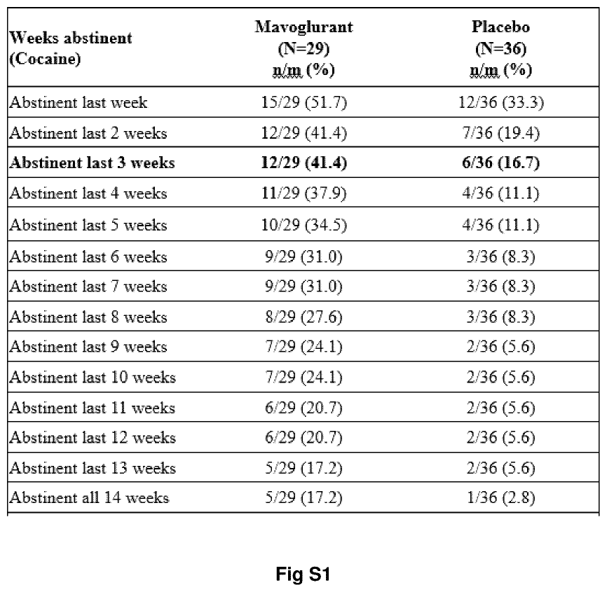

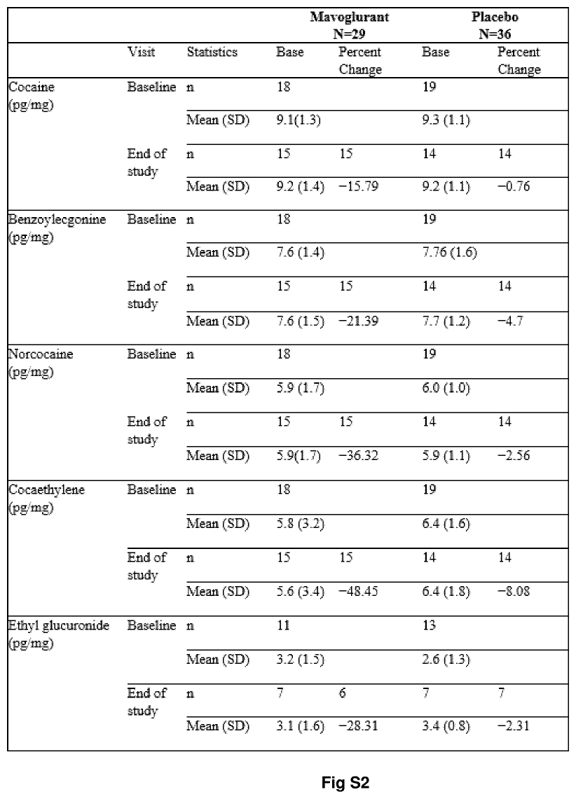

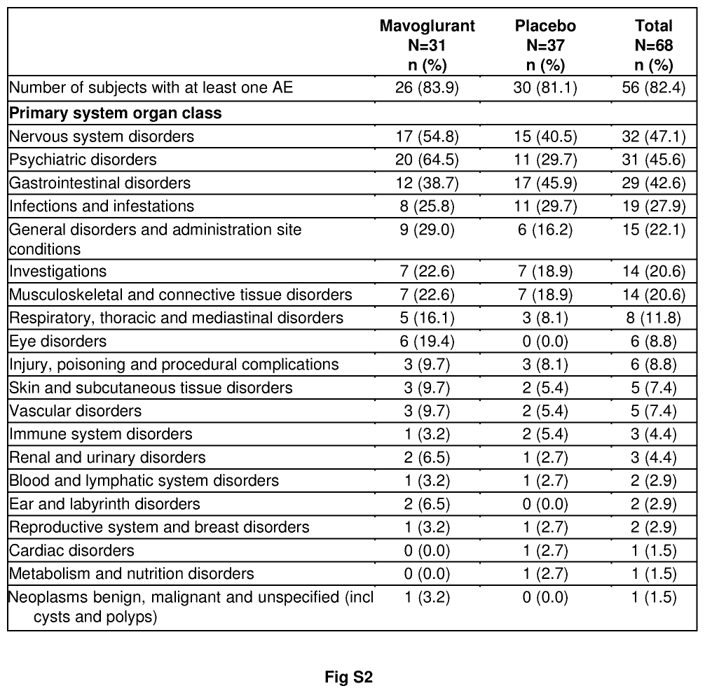

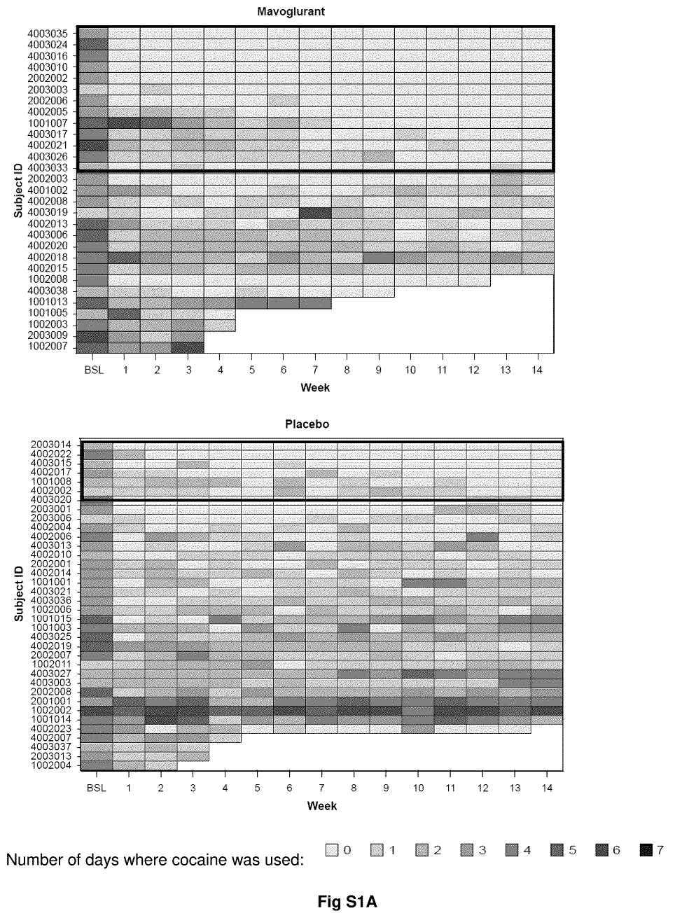

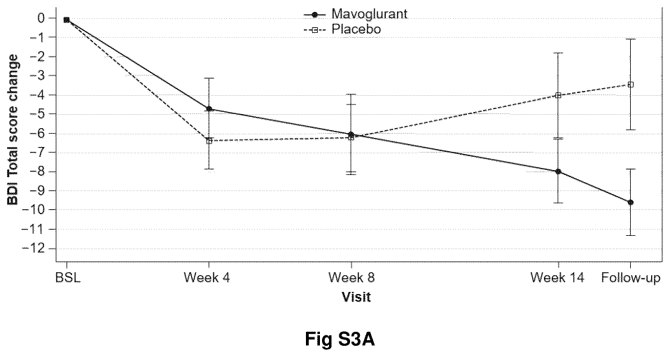

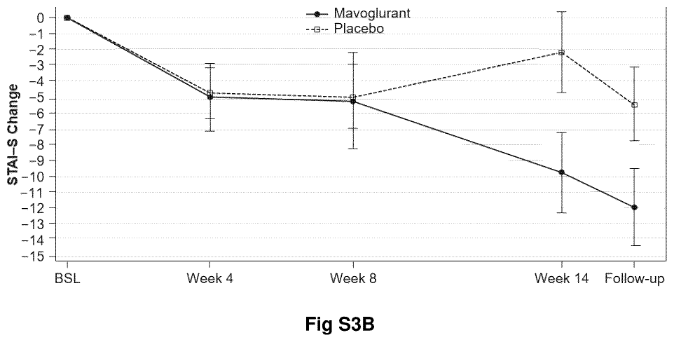

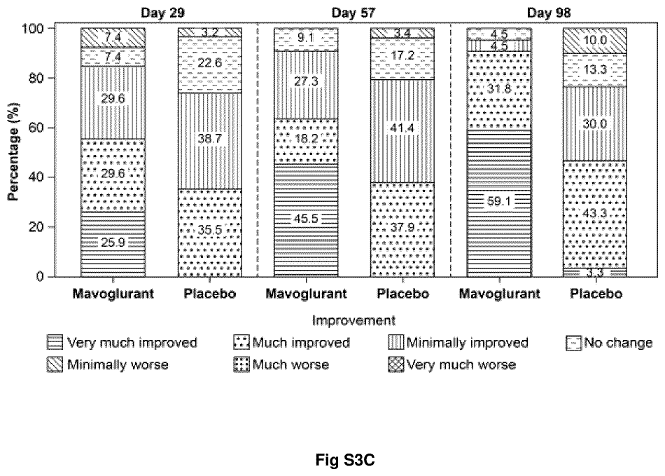

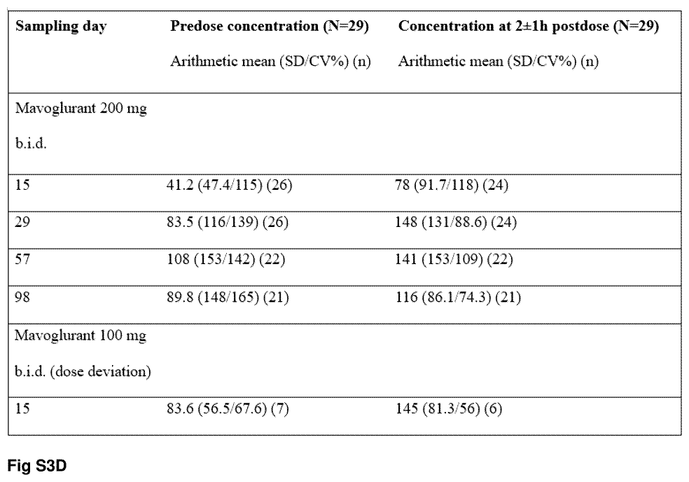

*ANCOVA model with treatment and country as a factors and baseline cocaine use as covariates.

† Linear mixed model for repeated measures with treatment, time, and treatment by time interaction as fixed effects and baseline alcohol use as a covariate. An unstructured covariance matrix was used.

CI=confidence interval; SE=standard error.

FIG. S1. Cumulative proportion of patients abstinent from cocaine by treatment and number of weeks. N=total number of subjects in the intervention group, n=number of abstinent subjects at prespecified time point, m=number of subjects evaluable. A subject absent at the visit is non abstinent.

FIG. S2. Summary of log-transformed hair drug test for cocaine, benzoylecgonine, norcocaine, cocaethylene, and ethyl glucuronide. There were no patients below or above LLOQ and ULOQ for the above-mentioned compounds. The repeated measurements were not taken into account for the calculation of the statistics. Values below LLOQ were imputed as LLOQ/2 and values above ULOQ were imputed as ULOQ for the analysis and prior to log-transformation. N=total number of subjects in the intervention group, n=all observations non-missing, including censored data (values below the LLOQ and values above the ULOQ).

FIG. S2. Adverse events by system organ class (SOC). N=total number of subjects in the intervention group, n=number of subjects with adverse events. A subject with multiple adverse events is counted only once in at least one adverse event row. A subject with multiple adverse events within a primary SOC is counted only once for that SOC and treatment.

FIG. S1A. Effect of mavoglurant on weekly cocaine use (by TLFB): Heat map of the weekly number of cocaine use days. N=29 (Mavoglurant), N=36 (Placebo). N=total number of subjects in the intervention group. Black box shows subjects with minimal cocaine usage.

FIG. S3A. Effect of mavoglurant on depressive symptoms. Mean (SE) change from baseline in Beck Depression Inventory (BDI) total score by visit and treatment.

FIG. S3B. Effect of mavoglurant on anxiety. Mean (SE) change from baseline in State-trait Anxiety Inventory (STAI). BSL=Baseline.

FIG. S3C. Effect of mavoglurant on global functioning: Clinical Global Impression (CGI) improvement. Wilcoxon test: p<0.0001.

FIG. S3D. Pharmacokinetics. b.i.d.=twice a day; CV=coefficient of variance; N=total number of patients in the intervention group; SD=standard deviation; n=number of subjects.

### DETAILED DESCRIPTION OF THE INVENTION

Mavoglurant may be an ideal candidate for treating patients diagnosed with substance use disorder, wherein the substance is an amphetamine-type stimulant, having therapeutic advantages for said patient population, such as one or more of the following:
i) promoting amphetamine-type stimulant abstinence, for example, compared to placebo, for example by maintaining abstinence or by reducing the amount or frequency of amphetamine-type stimulant use, for example as assessed by urinalysis (e.g. by measuring metabolites of the amphetamine-type stimulant in urine, such as metabolites of methamphetamine) or as assessed by using self-reported amphetamine-type stimulant use with standardized tools like the Timeline Follow-Back self-report [e.g. Sobell, L.C., Sobell, M. B. (1996) Timeline Followback User's Guide: A Calendar Method for Assessing Alcohol and Drug Use, Addiction Research Foundation. Toronto, Ontario, Canada; J. Anal. Toxicolo., 2002, 26: 393-400];
ii) decreasing relapse into amphetamine-type stimulant use, for example, compared to placebo, for example it increases the time to relapse or the rates of patient relapse in a treatment program, such as a clinical trial;
iii) alleviating (e.g. by eliminating or by reducing intensity, duration or frequency), for example compared to placebo, one or more of symptoms associated with substance use disorder, wherein the substance is an amphetamine-type stimulant, selected from:
   a. depressive symptoms, for example as assessed from the Beck's Depression Inventory [Beck, A. T. et al., (1961) An inventory for measuring depression. Archives of General Psychiatry, 4, 561-571; Beck, A. T. et aL, (1988) Psychometric properties of the Beck Depression Inventory: Twenty-five years of evaluation. Clinical Psychology Review, 8(1), 77-100]; and
   b. anxiety symptoms, for example as assessed from the State-Trait Anxiety Inventory [Spielberger, C. D. (1989). State-Trait Anxiety Inventory: Bibliography (2 nd Ed.). Palo Alto, CA: Consulting Psychologists Press; Spielberger, C. D. et aL, (1983). Manual for the State-Trait Anxiety Inventory. Palo Alto, CA: Consulting Psychologists Press].
iv) increasing retention of patients in treatment, for example, compared to placebo, for example it increases the rates of patient retention in a treatment program, such as a clinical trial (e.g. as measured by patient attendance at scheduled clinic visits and/or time to dropout from clinical protocol);
v) it improves global functioning, for example as assessed from the Clinical Global Impression Scale-Severity (CGI-S) and Improvement (CGI-I) (Psychiatry, 2007, 4(7): 28-37); or
vi) it has a favorable therapeutic profile, such as a favorable safety profile or metabolic profile, for example a favorable profile in relation to psychiatric adverse events, genotoxicity, or cardiovascular adverse events (e.g. blood pressure, heart rate, electrocardiography parameters); for example, it has better therapeutic profile (e.g. fewer side effects, decreased off-target effects or decreased toxicity, such as decreased genotoxicity) compared to known therapeutic agent/s that have been tested in the treatment of substance use disorder, wherein the substance is amphetamine-type stimulant.

Embodiments of the present invention are:

### EMBODIMENTS (a)

1a. Mavoglurant, or a pharmaceutically acceptable salt thereof, for use in the treatment of substance use disorder, wherein the substance is an amphetamine-type stimulant.

2a. Mavoglurant, or a pharmaceutically acceptable salt thereof, for use in the reduction of substance use by a substance use disorder patient, wherein the substance is an amphetamine-type stimulant.

3a. Mavoglurant, or a pharmaceutically acceptable salt thereof, for use in a treatment to prevent relapse into substance use by a substance use disorder patient, wherein the substance is an amphetamine-type stimulant.

4a. Mavoglurant, or a pharmaceutically acceptable salt thereof, for use in a treatment to promote substance abstinence by a substance use disorder patient, wherein the substance is an amphetamine-type stimulant.

5a. Mavoglurant, or a pharmaceutically acceptable salt thereof, for use in the treatment of the symptoms of depression or anxiety associated with substance use disorder, wherein the substance is an amphetamine-type stimulant.

6a. Mavoglurant, or a pharmaceutically acceptable salt thereof, for use according to any one of embodiments 1a to 5a, wherein the method of amphetamine-type stimulant use is inhalation (i.e. smoking), intravenous injection, nasal insufflation (i.e. snorting) or oral ingestion of the amphetamine-type stimulant.

7a. Mavoglurant, or a pharmaceutically acceptable salt thereof, for use according to any one of embodiments 1a to 6a, wherein the substance use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.

8a. Mavoglurant, or a pharmaceutically acceptable salt thereof, for use according to any one of embodiments 1a to 7a, wherein the use is combined with standardized psychological treatment, for example, at individual or group level.

9a. Mavoglurant, or a pharmaceutically acceptable salt thereof, for use according to any one of embodiments 1a to 8a, wherein the use is combined with psychosocial or behavioral therapy or combination thereof, in particular contingency management based therapy.

10a. Mavoglurant, or a pharmaceutically acceptable salt thereof, for use according to embodiment 9a, wherein the psychosocial or the behavioral therapy is computer-assisted.

11a. Mavoglurant, or a pharmaceutically acceptable salt thereof, for use according to any one of embodiments 1a to 10a, wherein mavoglurant, or a pharmaceutically acceptable salt thereof, is administered in combination with suvorexant, bupropion or mirtazapine, or salts thereof, in particular mirtazapine or a salt thereof.

12a. Mavoglurant, or a pharmaceutically acceptable salt thereof, for use according to any one of embodiments 1a to 11a, wherein mavoglurant, or a pharmaceutically acceptable salt thereof, is administered in combination with a further active agent, for example wherein the further active agent is selected from the group consisting of an antidepressant, an antipsychotic and an anxiolytic.

13a. Mavoglurant, or a pharmaceutically acceptable salt thereof, for use according to any one of embodiments 1a to 12a, wherein the patient has a genetic variation associated with a substance use disorder.

14a. Mavoglurant, or a pharmaceutically acceptable salt thereof, for use according to any one of embodiments 1a to 13a, wherein mavoglurant, or a pharmaceutically acceptable salt thereof, is administered in an immediate-release form or a modified-release form.

15a. Mavoglurant, or a pharmaceutically acceptable salt thereof, for use according to any one of embodiments 1a to 14a, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.

16a. Mavoglurant, or a pharmaceutically acceptable salt thereof, for use according to embodiment 15a, wherein mavoglurant, or a pharmaceutically acceptable salt thereof, is administered with food.

17a. Mavoglurant, or a pharmaceutically acceptable salt thereof, for use according to embodiment 15a or 16a, wherein mavoglurant, or a pharmaceutically acceptable salt thereof, is administered in the morning and in the evening separated by a 12 hour interval.

18a. Mavoglurant, or a pharmaceutically acceptable salt thereof, for use according to any of embodiments 1a to 17a, wherein the substance use disorder is associated with binge drinking or alcohol use disorder.

19a. Mavoglurant, or a pharmaceutically acceptable salt thereof, for use according to any of embodiments 1a to 18a, wherein mavoglurant, or a pharmaceutically acceptable salt thereof, is provided in the form of a pharmaceutical composition.

20a. Mavoglurant, or a pharmaceutically acceptable salt thereof, for use according to any of embodiments 1a to 18a, wherein mavoglurant, or a pharmaceutically acceptable salt thereof, is provided in the form of a pharmaceutical combination.

21a. Mavoglurant, or a pharmaceutically acceptable salt thereof, for use according to any one of embodiments 1a to 20a, wherein the amphetamine-type stimulant is selected from the group consisting of amphetamine, dextroamphetamine, methamphetamine, methylphenidate, methcathinone, fenetylline, ephedrine, pseudoephedrine, methylphenidate, 3,4-methylenedioxymethamphetamine, diethylpropion, lisdexamfetamine, phentermine, benzphetamine, mephentermine, midoamfetamine, tenamphetamine, 4-hydroxyamphetamine and mephedrone, in particular methamphetamine.

22a. Mavoglurant, or a pharmaceutically acceptable salt thereof, for use in the treatment according to any one of embodiments 1a to 20a, wherein the amphetamine-type stimulant is methamphetamine.

23a. Mavoglurant, or a pharmaceutically acceptable salt thereof, for use in the treatment according to any one of embodiments 1a to 22a, wherein the substance use disorder is mild substance use disorder, moderate substance use disorder or severe substance use disorder.

### EMBODIMENTS (b)

1b. Use of mavoglurant, or a pharmaceutically acceptable salt thereof, for the manufacture of a medicament for the treatment of substance use disorder, wherein the substance is an amphetamine-type stimulant.

2b. Use of mavoglurant, or a pharmaceutically acceptable salt thereof, for the manufacture of a medicament for a treatment in the reduction of substance use by a substance use disorder patient, wherein the substance is an amphetamine-type stimulant.

3b. Use of mavoglurant, or a pharmaceutically acceptable salt thereof, for the manufacture of a medicament for a treatment to prevent relapse into substance use by a substance use disorder patient, wherein the substance is an amphetamine-type stimulant.

4b. Use of mavoglurant, or a pharmaceutically acceptable salt thereof, for the manufacture of a medicament for a treatment to promote substance abstinence by a substance use disorder patient, wherein the substance is an amphetamine-type stimulant.

5b. Use of mavoglurant, or a pharmaceutically acceptable salt thereof, for the manufacture of a medicament for the treatment of the symptoms of depression or anxiety associated with substance use disorder, wherein the substance is an amphetamine-type stimulant.

6b. Use of mavoglurant, or a pharmaceutically acceptable salt thereof, according to any one of embodiments 1b to 5b, wherein the method of amphetamine-type stimulant use is inhalation (i.e. smoking), intravenous injection, nasal insufflation (i.e. snorting) or oral ingestion of the amphetamine-type stimulant.

7b. Use of mavoglurant, or a pharmaceutically acceptable salt thereof, according to any one of embodiments 1b to 6b, wherein the substance use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.

8b. Use of mavoglurant, or a pharmaceutically acceptable salt thereof, according to any one of embodiments 1b to 7b, wherein the use is combined with standardized psychological treatment, for example, at individual or group level.

9b. Use of mavoglurant, or a pharmaceutically acceptable salt thereof, according to any one of embodiments 1b to 8b, wherein the use is combined with psychosocial or behavioral therapy or combination thereof, in particular contingency management based therapy.

10b. Use of mavoglurant, or a pharmaceutically acceptable salt thereof, according to embodiment 9b, wherein the psychosocial or the behavioral therapy is computer-assisted.

11b. Use of mavoglurant, or a pharmaceutically acceptable salt thereof, according to any one of embodiments 1b to 10b, wherein mavoglurant, or a pharmaceutically acceptable salt thereof, is administered in combination with suvorexant, bupropion or mirtazapine, or salts thereof, in particular mirtazapine or a salt thereof.

12b. Use of mavoglurant, or a pharmaceutically acceptable salt thereof, according to any one of embodiments 1b to 11b, wherein mavoglurant, or a pharmaceutically acceptable salt thereof, is administered in combination with a further active agent, for example wherein the further active agent is selected from the group consisting of an antidepressant, an antipsychotic and an anxiolytic.

13b. Use of mavoglurant, or a pharmaceutically acceptable salt thereof, according to any one of embodiments 1b to 12b, wherein the patient has a genetic variation associated with a substance use disorder.

14b. Use of mavoglurant, or a pharmaceutically acceptable salt thereof, according to any one of embodiments 1b to 13b, wherein mavoglurant, or a pharmaceutically acceptable salt thereof, is administered in an immediate-release form or a modified-release form.

15b. Use of mavoglurant, or a pharmaceutically acceptable salt thereof, according to any one of embodiments 1b to 14b, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.

16b. Use of mavoglurant, or a pharmaceutically acceptable salt thereof, according to embodiment 15b, wherein mavoglurant, or a pharmaceutically acceptable salt thereof, is administered with food.

17b. Use of mavoglurant, or a pharmaceutically acceptable salt thereof, according to embodiment 15b or 16b, wherein mavoglurant, or a pharmaceutically acceptable salt thereof, is administered in the morning and in the evening separated by a 12 hour interval.

18b. Use of mavoglurant, or a pharmaceutically acceptable salt thereof, according to any of embodiments 1b to 17b, wherein the substance use disorder is associated with binge drinking or alcohol use disorder.

19b. Use of mavoglurant, or a pharmaceutically acceptable salt thereof, according to any of embodiments 1b to 18b, wherein mavoglurant, or a pharmaceutically acceptable salt thereof, is provided in the form of a pharmaceutical composition.

20b. Use of mavoglurant, or a pharmaceutically acceptable salt thereof, according to any of embodiments 1b to 18b, wherein mavoglurant, or a pharmaceutically acceptable salt thereof, is provided in the form of a pharmaceutical combination.

21b. Use of mavoglurant, or a pharmaceutically acceptable salt thereof, according to any one of embodiments 1b to 20b, wherein the amphetamine-type stimulant is selected from the group consisting of amphetamine, dextroamphetamine, methamphetamine, methylphenidate, methcathinone, fenetylline, ephedrine, pseudoephedrine, methylphenidate, 3,4-methylenedioxymethamphetamine, diethylpropion, lisdexamfetamine, phentermine, benzphetamine, mephentermine, midoamfetamine, tenamphetamine, 4-hydroxyamphetamine and mephedrone, in particular methamphetamine.

22b. Use of mavoglurant, or a pharmaceutically acceptable salt thereof, according to any one of embodiments 1b to 20b, wherein the amphetamine-type stimulant is methamphetamine.

23b. Use of mavoglurant, or a pharmaceutically acceptable salt thereof, according to any one of embodiments 1b to 22b, wherein the substance use disorder is mild substance use disorder, moderate substance use disorder or severe substance use disorder.

### EMBODIMENTS (c)

1c. A method for treating substance use disorder, wherein the substance is an amphetamine-type stimulant, in a patient in need thereof, comprising administering to said patient an effective amount of mavoglurant, or a pharmaceutically acceptable salt thereof.

2c. A method for the reduction of substance use by a substance use disorder patient, in need thereof, wherein the substance is an amphetamine-type stimulant, comprising administering to said patient an effective amount of mavoglurant, or a pharmaceutically acceptable salt thereof.

3c. A method for preventing relapse into substance use by a substance use disorder patient, in need thereof, wherein the substance is an amphetamine-type stimulant, comprising administering to said patient an effective amount of mavoglurant, or a pharmaceutically acceptable salt thereof.

4c. A method for the promotion of substance abstinence by a substance use disorder patient, in need thereof, wherein the substance is an amphetamine-type stimulant, comprising administering to said patient an effective amount of mavoglurant, or a pharmaceutically acceptable salt thereof.

5c. A method for treating the symptoms of depression or anxiety associated with substance use disorder, wherein the substance is an amphetamine-type stimulant, comprising administering to said patient an effective amount of mavoglurant, or a pharmaceutically acceptable salt thereof.

6c. The method according to any one of embodiments 1c to 5c, wherein the method of amphetamine-type stimulant use is inhalation (i.e. smoking), intravenous injection, nasal insufflation (i.e. snorting) or oral ingestion of the amphetamine-type stimulant.

7c. The method according to any one of embodiments 1c to 6c, wherein the substance use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.

8c. The method according to any one of embodiments 1c to 7c, wherein the use is combined with standardized psychological treatment, for example, at individual or group level.

9c. The method according to any one of embodiments 1c to 8c, wherein the use is combined with psychosocial or behavioral therapy or combination thereof, in particular contingency management based therapy.

10c. The method according to embodiment 9c, wherein the psychosocial or the behavioral therapy is computer-assisted.

11c. The method according to any one of embodiments 1c to 10c, wherein mavoglurant, or a pharmaceutically acceptable salt thereof, is administered in combination with suvorexant, bupropion or mirtazapine, or salts thereof, in particular mirtazapine or a salt thereof.

12c. The method according to any one of embodiments 1c to 11c, wherein mavoglurant, or a pharmaceutically acceptable salt thereof, is administered in combination with a further active agent, for example wherein the further active agent is selected from the group consisting of an antidepressant, an antipsychotic and an anxiolytic.

13c. The method according to any one of embodiments 1c to 12c, wherein the patient has a genetic variation associated with a substance use disorder.

14c. The method according to any one of embodiments 1c to 13c, wherein mavoglurant, or a pharmaceutically acceptable salt thereof, is administered in an immediate-release form or a modified-release form.

15c. The method according to any one of embodiments 1c to 14c, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.

16c. The method according to embodiment 15c, wherein mavoglurant, or a pharmaceutically acceptable salt thereof, is administered with food.

17c. The method according to embodiment 15c or 16c, wherein mavoglurant, or a pharmaceutically acceptable salt thereof, is administered in the morning and in the evening separated by a 12 hour interval.

18c. The method according to any of embodiments 1c to 17c, wherein the substance use disorder is associated with binge drinking or alcohol use disorder.

19c. The method according to any of embodiments 1c to 18c, wherein mavoglurant, or a pharmaceutically acceptable salt thereof, is provided in the form of a pharmaceutical composition.

20c. The method according to any of embodiments 1c to 18c, wherein mavoglurant, or a pharmaceutically acceptable salt thereof, is provided in the form of a pharmaceutical combination.

21c. The method according to any one of embodiments 1c to 20c, wherein the amphetamine-type stimulant is selected from the group consisting of amphetamine, dextroamphetamine, methamphetamine, methylphenidate, methcathinone, fenetylline, ephedrine, pseudoephedrine, methylphenidate, 3,4-methylenedioxymethamphetamine, diethylpropion, lisdexamfetamine, phentermine, benzphetamine, mephentermine, midoamfetamine, tenamphetamine, 4-hydroxyamphetamine and mephedrone, in particular methamphetamine.

22c. The method according to any one of embodiments 1c to 20c, wherein the amphetamine-type stimulant is methamphetamine.

23c. The method according to any one of embodiments 1c to 22c, wherein the substance use disorder is mild substance use disorder, moderate substance use disorder or severe substance use disorder.

General Terms

The term “substance use disorder”, as used herein above or below, refers to “stimulant use disorder”, for example defined with reference to diagnostic criteria such as DSM-5 criteria (i.e. according to the Diagnostic and Statistical Manual of Mental Disorders. 5^{th} Edition, Washington, DC: American Psychiatric Association, 2013), the entire contents of which, in particular contents of the section on “stimulant use disorder”, are incorporated herein by reference. In one embodiment, the expression “substance use disorder, wherein the substance is an amphetamine-type stimulant” refers to “stimulant use disorder, wherein the stimulant is an amphetamine-type stimulant”, in a particular embodiment it refers to “methamphetamine use disorder”. In one embodiment, the expression “substance use disorder, wherein the substance is an amphetamine-type stimulant (herein below referred as “stimulant”)” is defined as a pattern of stimulant use leading to clinically significant impairment or distress, as manifested by at least two of the following, occurring within a 12-month period:
1. The stimulant is often taken in larger amounts or over a longer period than was intended.
2. There is a persistent desire or unsuccessful efforts to cut down or control stimulant use.
3. A great deal of time is spent in activities necessary to obtain the stimulant, use the stimulant, or recover from its effects.
4. Craving, or a strong desire or urge to use the stimulant
5. Recurrent stimulant use resulting in a failure to fulfill major role obligations at work, school, or home.
6. Continued stimulant use despite having persistent or recurrent social or interpersonal problems caused or exacerbated by the effects of the stimulant.
7. Important social, occupational, or recreational activities are given up or reduced because of stimulant use.
8. Recurrent stimulant use in situations in which it is physically hazardous.
9. Stimulant use is continued despite knowledge of having a persistent or recurrent physical or psychological problem that is likely to have been caused or exacerbated by the stimulant.
10. Tolerance, as defined by either of the following:
   1. A need for markedly increased amounts of the stimulant to achieve intoxication or desired effect.
   2. A markedly diminished effect with continued use of the same amount of the stimulant.
   Note: This criterion is not considered to be met for those taking stimulant medications solely under appropriate medical supervision, such as medications for attention-deficit/hyperactivity disorder or narcolepsy.
11. Withdrawal, as manifested by either of the following:
   a) The characteristic withdrawal syndrome for the stimulant:
      i) cessation of (or reduction in) prolonged stimulant use;
      ii) dysphoric mood and two (or more) of the following physiological changes, developing within a few hours to several days after the cessation of (or reduction in) stimulant use: fatigue; vivid, unpleasant dreams; insomnia or hypersomnia; increased appetite; psychomotor retardation or agitation.
   b) The stimulant is taken to relieve or avoid withdrawal symptoms.
   Note: This criterion is not considered to be met for those taking stimulant medications solely under appropriate medical supervision, such as medications for attention-deficit/hyperactivity disorder or narcolepsy.

“Substance use disorder” may be separated into the following three categories: mild (e.g. presence of 2 to 3 symptoms, defined with reference to DSM-5 criteria), moderate (e.g. presence of 4 to 5 symptoms, defined with reference to DSM-5 criteria) and severe (e.g. presence of 6 or more symptoms, defined with reference to DSM-5 criteria), wherein as stated hereinabove the term “substance use disorder”, as used herein, refers to “stimulant use disorder”, as defined herein, in particular “substance use disorder, wherein the substance is an amphetamine-type stimulant”, such as “methamphetamine use disorder”. In one embodiment “substance use disorder”, as used herein, refers to “mild substance use disorder”, “moderate substance use disorder” and “severe substance use disorder”, wherein as stated hereinabove the term “substance use disorder”, as used herein, refers to “stimulant use disorder”, as defined herein, in particular “substance use disorder, wherein the substance is an amphetamine-type stimulant”, such as “methamphetamine use disorder”.

The term “substance use disorder patient” refers to a patient diagnosed with substance use disorder, as defined herein, namely, wherein as stated hereinabove the term “substance use disorder”, as used herein, refers to “stimulant use disorder”, as defined herein, in particular “substance use disorder, wherein the substance is an amphetamine-type stimulant”, such as “methamphetamine use disorder”.

In one embodiment, the term “substance use disorder patient” refers to a patient diagnosed with substance use disorder, wherein the substance is an amphetamine-type stimulant (e.g. methamphetamine) who is in abstinence from such a substance, for example, for at least 1 day, such as 3 days or more. The term “substance use disorder patient in abstinence” refers to a patient diagnosed with substance use disorder [i.e. as defined herein, namely, wherein as stated hereinabove the term “substance use disorder”, as used herein, refers to “stimulant use disorder”, as defined herein, in particular “substance use disorder, wherein the substance is an amphetamine-type stimulant”, such as “methamphetamine use disorder”] in abstinence from amphetamine-type stimulant for a period, for example, for at least 1 day. The term “substance use disorder associated with binge drinking” refers to a patient who is diagnosed with substance use disorder, as defined herein [i.e. namely, wherein as stated hereinabove the term “substance use disorder”, as used herein, refers to “stimulant use disorder”, as defined herein in particular “substance use disorder, wherein the substance is an amphetamine-type stimulant”, such as “methamphetamine use disorder”], and who is an abuser of alcohol (i.e. a heavy drinker). As explained at http://drugabuse.com/library/alcohol-abuse/, abusers of alcohol may not drink on a consistent basis, for example, they may only drink once a week, but, when drinking, they may drink heavily, which will cause problems, such as suffering from alcohol intoxication. For the sake of clarity, herein, an abuser of alcohol is not an alcohol use disorder patient (i.e. does not meet criteria for alcohol use disorder as defined with reference to DSM-5 criteria). The term “heavy drinker” refers, for example, to someone with a heavy alcohol use pattern. According to the National Institute on Alcohol Abuse and Alcoholism (NIAAA), the Substance Abuse and Mental Health Services Administration (SAMHSA) defines “heavy alcohol use” as binge drinking on 5 or more days in the past month. NIAAA defines binge drinking as a pattern of drinking that brings blood alcohol concentration (BAC) levels to 0.08 g/dL. This typically occurs after 4 alcoholic drinks for women and 5 alcoholic drinks for men—in about 2 hours. The Substance Abuse and Mental Health Services Administration (SAMHSA), defines “binge drinking” as 5 or more alcoholic drinks for males or 4 or more alcoholic drinks for females on the same occasion (i.e., at the same time or within a couple of hours of each other) on at least 1 day in the past month. The term “alcohol”, as used herein, for example in relation to “drinks”, “alcoholic drinks” or “drinking”, refers to ethyl alcohol (i.e. ethanol). The term “drinking”, “drinks” or “alcoholic drinks”, as used herein, is understood in the context of “standard drinks”, such as spirits or blends that are intended for human consumption, wherein a “standard drink” equals 12 g ethanol.

The term “substance use disorder associated with alcohol use disorder” refers to a patient who is diagnosed with substance use disorder, as defined herein [i.e. namely, wherein as stated hereinabove the term “substance use disorder”, as used herein, refers to “stimulant use disorder”, as defined herein, in particular “substance use disorder, wherein the substance is an amphetamine-type stimulant”, such as “methamphetamine use disorder”], and who is also diagnosed with alcohol use disorder (i.e. it meets criteria for alcohol use disorder, for example as defined with reference to DSM-5 criteria i.e. according to the Diagnostic and Statistical Manual of Mental Disorders. 5^{th} Edition, Washington, DC: American Psychiatric Association, 2013, the entire contents of which, in particular contents of the section on “alcohol use disorder”, are incorporated herein by reference).

The term “amphetamine-type stimulant”, as used herein, refers, for example, to compounds comprising a phenylethylamine structure, such as amphetamine, analogs or derivatives of amphetamine, as well as compounds that are structurally different but have similar effects, such as methylphenidate, methcathinone, fenetylline, ephedrine, pseudoephedrine, methylphenidate and 3,4-methylenedioxymethamphetamine. In embodiments the amphetamine-type stimulant is, for example, selected from the group consisting of amphetamine, dextroamphetamine, methamphetamine, methylphenidate, methcathinone, fenetylline, ephedrine, pseudoephedrine, methylphenidate, 3,4-methylenedioxymethamphetamine, diethylpropion, lisdexamfetamine, phentermine, benzphetamine, mephentermine, midoamfetamine, tenamphetamine, 4-hydroxyamphetamine or mephedrone, in particular methamphetamine.

The term “amphetamine-type stimulant use”, as used herein, refers to amphetamine-type stimulant consumption.

The term “reducing substance use” or “reduction of substance use”, as used herein, refers to, for example, reducing the amount or frequency of substance use, wherein the substance is an amphetamine-type stimulant, as defined herein, for example as assessed by urinalysis (e.g. by measuring metabolites of an amphetamine-type stimulant in urine, such as metabolites of methamphetamine) or as assessed by using self-reported amphetamine-type stimulant (e.g. methamphetamine) use with standardized self-report tools like the Timeline Follow-Back self-report (e.g. Sobell LC, Sobell MB, 1996, Timeline FollowBack user's guide: A calendar method for assessing alcohol and drug use. Addiction Research Foundation, Toronto, Ontario, Canada; J. Anal. Toxicolo., 2002, 26: 393-400).

The term “substance abstinence” or “in abstinence from substance”, as used herein, refers to, for example, not taking an amphetamine-type stimulant, as defined herein. The term “promoting substance abstinence” or “promotion of substance abstinence”, as used herein, refers to, for example, help maintaining abstinence from amphetamine-type stimulant use, as defined herein, in particular after at least 1 day of not taking amphetamine-type stimulants, as defined herein, for example maintaining abstinence from amphetamine-type stimulant use for a period of, for example, at least 1 week, 2 weeks, 3 weeks, 1 month, 3 months, 6 months or more, in particular at least 1 week or more, such as 2 weeks.

The term “relapse into substance use” or “relapse into substance consumption”, as used herein, refers to, for example, amphetamine-type stimulant intake (i.e. taking an amphetamine-type stimulant) following a period of amphetamine-type stimulant abstinence, for example following a period of amphetamine-type stimulant abstinence of at least 1 day or more, such as 3 days, 1 week, 2 weeks, 3 weeks, 1 month, 3 months, 6 months or more.

The term “preventing relapse into substance use” or “preventing relapse into substance consumption”, as used herein, refers to, for example, the prevention of amphetamine-type stimulant intake by a substance use disorder patient, as defined herein, after the patient has stopped the intake of amphetamine-type stimulants, in particular after 1 day or more of not taking amphetamine-type stimulants. In some embodiments, the term encompasses the permanent stoppage of amphetamine-type stimulant intake. In other embodiments, the term encompasses a delay in the resumption of amphetamine-type stimulant intake as compared to the time to resumption by a subject that is not administered a compound of the invention. The delay in resumption can be, e.g., days (e.g., 2, 3, 4, 5, 6, 7 days), weeks (e.g., 1, 2, 3 weeks), months (e.g., 1, 2, 3, 4, 5, 6 months), or longer.

The term “antidepressant”, as used herein, refers to an active ingredient commonly used to treat depression, such as a serotonin reuptake inhibitor (SSRI, e.g., fluoxetine, citalopram, sertraline, paroxetine, escitalopram, fluvoxamine, vilazodone, vortioxetine), a serotonin and norepinephrine reuptake inhibitor (SNRI, e.g., venlafaxine, duloxetine, desvenlafaxine, milnacipran, levomilnacipran), bupropion, a tricyclic antidepressant (e.g. amitriptyline, nortriptyline, doxepin, desipramine, imipramine, protriptyline, trimipramine, clomipramine), a tetracyclic antidepressant (e.g. maprotiline, mianserin, mirtazapine, setiptiline), or a monoamine oxidase inhibitor (MAOI, e.g. isocarboxazid, phenelzine, selegiline, tranylcypromine). In one embodiment, the antidepressant is selected from the group consisting of a serotonin reuptake inhibitor (SSRI, e.g., fluoxetine, citalopram, sertraline, paroxetine, escitalopram, fluvoxamine, vilazodone, vortioxetine), a serotonin and norepinephrine reuptake inhibitor (SNRI, e.g., venlafaxine, duloxetine, desvenlafaxine, milnacipran, levomilnacipran), bupropion, a tricyclic antidepressant (e.g. amitriptyline, nortriptyline, doxepin, desipramine, imipramine, protriptyline, trimipramine, clomipramine), a tetracyclic antidepressant (e.g. maprotiline, mianserin, mirtazapine, setiptiline), a monoamine oxidase inhibitor (MAOI, e.g. isocarboxazid, phenelzine, selegiline, tranylcypromine) and hypericum perforatum. In another embodiment, the antidepressant is selected from the group consisting of fluoxetine, citalopram, sertraline, paroxetine, escitalopram, fluvoxamine, vilazodone, vortioxetine, venlafaxine, duloxetine, desvenlafaxine, milnacipran, levomilnacipran, bupropion, amitriptyline, nortriptyline, doxepin, desipramine, imipramine, protriptyline, trimipramine, clomipramine, maprotiline, mianserin, mirtazapine, setiptiline, isocarboxazid, phenelzine, selegiline, tranylcypromine and hypericum perforatum; or salts thereof. In another embodiment, the antidepressant is selected from the group consisting of fluoxetine, citalopram, sertraline, paroxetine, escitalopram, fluvoxamine, vilazodone, vortioxetine, venlafaxine, duloxetine, desvenlafaxine, milnacipran, levomilnacipran, bupropion, amitriptyline, nortriptyline, doxepin, desipramine, imipramine, protriptyline, trimipramine, clomipramine, maprotiline, mianserin, mirtazapine, setiptiline, isocarboxazid, phenelzine, selegiline and tranylcypromine; or salts thereof.

The term “anxiolytic”, as used herein, refers to a drug that inhibits anxiety, such as benzodiazepines (e.g. alprazolam, bromazepam, chlordiazepoxide, clonazepam, clorazepate, diazepam, flurazepam, lorazepam, oxazepam, temazepam, triazolam) or antihistamines (e.g. hydroxyzine). In one embodiment, the anxiolytic is selected from the group consisting of alprazolam, bromazepam, chlordiazepoxide, clonazepam, clorazepate, diazepam, flurazepam, lorazepam, oxazepam, temazepam, triazolam, and hydroxyzine; or salts thereof.

The term “antipsychotic”, as used herein, refers to a neuroleptic drug used to treat a psychotic disorder, such as schizophrenia. In one embodiment, the antipsychotic is, for example, selected from the group consisting of a typical antipsychotic and an atypical antipsychotic. In another embodiment, the antipsychotic is a typical antipsychotic. In yet another embodiment, the antipsychotic is an atypical antipsychotic. The term “typical antipsychotic”, as used herein, refers to a first-generation antipsychotic, for example selected from the group consisting of a butyrophenone (e.g., haloperidol), a diphenylbutylpiperidine (e.g., pimozide), a phenothiazine (e.g., chlorpromazine, fluphenazine, perphenazine, prochlorperazine, trifluoperazine), and a thioxanthene (e.g., thiothixene). In one embodiment, the typical antipsychotic is selected from the group consisting of haloperidol, pimozide, chlorpromazine, fluphenazine, perphenazine, prochlorperazine, trifluoperazine, and thiothixene; or salts thereof. The term “atypical antipsychotic”, as used herein, refers to a second-generation antipsychotic, for example selected from the group consisting of a benzamide (e.g., sultopride), a benzisoxazole/benzisothiazole (e.g., lurasidone, paliperidone, risperidone), a phenylpiperazine/quinolinone (e.g., aripiprazole, brexpiprazole, cariprazine) a tricyclic (e.g., clozapine, olanzapine, quetiapine, asenapine, zotepine). In one embodiment, the atypical antipsychotic is selected from the group consisting of sultopride, lurasidone, paliperidone, risperidone, brexpiprazole, cariprazine, clozapine, olanzapine, quetiapine, asenapine and zotepine; or salts thereof. In particulat, the antipsychotic is selected from the group consisting of risperidone, aripiprazole and haloperidol.

The term “psychosocial or behavioral therapy”, as used herein, refers to, but not limited to, cognitive behavioral therapy (e.g. as described in Arch. Gen. Psychiatry 1999; 56:493-502), interpersonal therapy (e.g. as described in Psychol Addict Behav2009; 23(1): 168-174), contingency management based therapy (e.g. as described in Psychol Addict Behav 2009; 23(1): 168-174; in J. Consul. Clin. Psychol. 2005; 73(2): 354-59; or in Case Reports in Psychiatry, Vol. 2012, Article ID 731638), community reinforcement approach based therapy (e.g. as described in Drug Alcohol Depend 2004; 74:1-13), motivational interviewing based therapy (e.g. as described in J. Consul. Clin. Psychol. 2001; 69(5): 858-62), motivational enhancement based therapy (e.g. as described in Drug Alcohol Depend 2007, 91:97-101) or meditation based therapy, such as transcendental meditation based therapy (e.g. as described in Addiction 2004; 99(7):862-874 or J. Consul. Clin. Psychol. 2000; 68(3): 515-52); in particular contingency management based therapy.

The term “standardized psychological treatment” or “standardized psychological support”, as used herein, refers to standard counselling sessions, for example once a week, in particular counselling focused on amphetamine-type stimulant consumption.

The term “computer-assisted” in the expression “the psychosocial or the behavioral therapy is computer-assisted”, as used herein, refers to psychosocial or behavioral therapy comprising the use of electronic tools such as online tools, smartphones, wireless devices or health Apps. In one embodiment, the term “computer-assisted” in the expression “the psychosocial or the behavioral therapy is computer-assisted”, as used herein, is to be understood as “computer-implemented” (i.e. the psychosocial or the behavioral therapy is computer-implemented).

The term “administered with food” refers to, for example, any food product, solid or liquid, with caloric content. The dosage of the mavoglurant, or pharmaceutically acceptable salt thereof, may be administered to a subject, for example, between thirty minutes prior to eating food, to, for example, one hour after consumption. In particular, administration of mavoglurant, or pharmaceutically acceptable salt thereof, occurs immediately after consuming food up to about thirty minutes after consumption.

The term “genetic variation” refers to a change in a gene sequence relative to a reference sequence (e.g., a commonly-found and/or wild-type sequence). Genetic variation may be recombination events or mutations such as substitution/deletion/insertion events like point and splice site mutations. In one embodiment, the genetic variation is a genetic variation in mGluR5.

The term “treat” “treating” “treatment” or “therapy”, as used herein, means obtaining beneficial or desired results, for example, clinical results. Beneficial or desired results can include, but are not limited to, alleviation of one or more symptoms of substance use disorder patients, as defined herein, such as anxiety symptoms or depression symptoms associated with substance use disorder, as defined herein, in particular by a substance use disorder patient, as defined herein, in abstinence from amphetamine-type stimulants, as herein defined. One aspect of the treatment is, for example, that said treatment should have a minimal adverse effect on the patient, e.g. the agent used should have a high level of safety, for example without producing the side effects of previously known treatment regimens. The term “alleviation”, for example in reference to a symptom of a condition, as used herein, refers to reducing at least one of the frequency and amplitude of a symptom of a condition in a patient.

As used herein, the term “subject” refers to a mammalian organism, preferably a human being (male or female).

As used herein, the term “patient” refers to a subject who is diseased and would benefit from the treatment.

As used herein, a subject is “in need of” a treatment if such a subject (patient) would benefit biologically, medically or in quality of life from such a treatment.

The term “pharmaceutical composition” is defined herein to refer to a mixture or solution containing at least one active ingredient or therapeutic agent to be administered to a subject, in order to treat a particular condition (i.e. disease, disorder or condition or at least one of the clinical symptoms thereof) affecting the subject.

As used herein, the term “pharmaceutically acceptable excipient” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 22^{nd} Ed. Mack Printing Company, 2013, pp. 1049-1070). Except insofar as any conventional carrier is incompatible with the active ingredient, its use in the therapeutic or pharmaceutical compositions is contemplated.

The terms “drug”, “active substance”, “active ingredient”, “pharmaceutically active ingredient”, “active agent” or “therapeutic agent” are to be understood as meaning a compound in free form or in the form of a pharmaceutically acceptable salt, in particular compounds of the type specified herein. In particular, reference to mavoglurant, or a pharmaceutically acceptable salt thereof, in combination with a further active agent, as used herein (e.g. in any of embodiments herein above, or in any of the claims, herein below), refers to mavoglurant in combination with at least one further active agent, for example selected from the group consisting of an antidepressant, an antipsychotic and an anxiolytic.

The term “immediate release form” refers to a pharmaceutical composition designed to release the active substance immediately upon in vivo administration.

The term “modified release form” refers to a pharmaceutical composition which releases the active substance not immediately, but offers a sustained, retard, continuous, gradual, prolonged or pulsatile release and therefore alters drug plasma levels distinctively versus an immediate release form. The term “modified release form” encompasses forms that are described as controlled-release form, sustained-release form, extended-release form, and long-acting form; in particular a sustained-release form.

The term “combination” or “pharmaceutical combination” refers to either a fixed combination in one unit dosage form (e.g., capsule, tablet, caplets or particulates), non-fixed combination, or a kit of parts for the combined administration where a compound of the present invention and one or more combination partner (e.g. another drug as specified herein, also referred to as further “pharmaceutical active ingredient”, “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. the compound of the present invention and one or more combination partners, are both administered to a patient simultaneously in the form of a single entity or dosage. The term “non-fixed combination” means that the active ingredients, e.g. a compound of the present invention and one or more combination partners, are both administered to a patient as separate entities either simultaneously or sequentially with no specific time limits, wherein such administration provides therapeutically effective levels of the two compounds in the body of the patient.

The compound of the present invention may be administered separately, by the same or different route of administration, or together in the same pharmaceutical composition as the other agents. In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

As used herein, the term “a,” “an,” “the” and similar terms used in the context of the present invention (especially in the context of the claims) are to be construed to cover both the singular and plural unless otherwise indicated herein or clearly contradicted by the context.

The use of any and all examples, or exemplary language (e.g. “such as”) provided herein is intended merely to better illuminate the invention and does not pose a limitation on the scope of the invention otherwise claimed.

As used herein, the compound of the invention, alternatively named Compound (I), as used herein above and below, is the mGluR5 antagonist (-)-(3aR,4S,7aR)-4-Hydroxy-4-m-tolylethynyl-octahydro-indole-1-carboxylic acid methyl ester, also named (-)-(3aR,4S,7aR)-4-Hydroxy-4-[2-(3-methylphenyl)ethynyl]perhydroindole-1-carboxylic acid methyl ester, also known as mavoglurant, of formula:

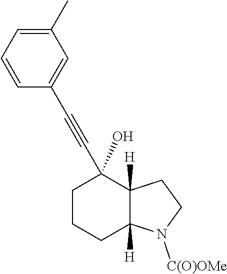

which can be e.g. prepared as described in WO2003/047581, e.g., in Example 1, or as described in WO2010/018154. WO2003/047581, which is incorporated herein by reference, also describes its in-vitro biological data, as per page 7. As used herein, “mavoglurant” refers to the free form, and any reference to “a pharmaceutically acceptable salt thereof” refers to a pharmaceutically acceptable acid addition salt thereof. As used herein, the term “mavoglurant, or a salt thereof, such as a pharmaceutically acceptable salt thereof”, as used in the context of the present invention (especially in the context of the any of the embodiments, above or below, and the claims) is thus to be construed to cover both the free form and a pharmaceutically acceptable salt thereof, unless otherwise indicated herein.

In one embodiment, Compound (I) is also intended to represent isotopically labeled forms. Isotopically labeled compounds have structures depicted by the formula above except that one or more atoms are replaced by an atom having a selected atomic mass or mass number. Isotopes that can be incorporated into the compound of the invention include, for example, isotopes of hydrogen, namely the compound of formula:

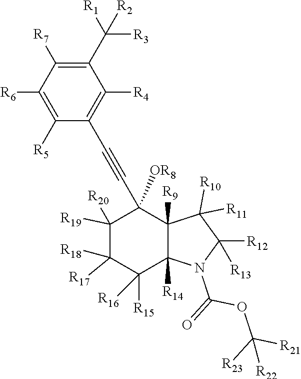

wherein each R₁, R₂, R₃, R₄, R₅, R₆, R₇, R₈, R₉, R₁₀, R₁₁, R₁₂, R₁₃, R₁₄, R₁₅, R₁₆, R₁₇, R₁₅, R₁₉, R₂₀, R₂₁, R₂₂ and R₂₃ is independently selected from H or deuterium; provided that there is at least one deuterium present in the compound. In other embodiments there are multiple deuterium atoms present in the compound.

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of the compound of the invention. The concentration of deuterium, may be defined by the isotopic enrichment factor. The term “isotopic enrichment factor” as used herein means the ratio between the isotopic abundance and the natural abundance of a specified isotope. If a substituent in the compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into the compound of the invention include isotopes of hydrogen, other than deuterium, carbon, nitrogen, oxygen, and fluorine such as ³H, ¹¹C, ¹³C, ¹⁴C, ¹⁵N, ¹⁸F respectively. Accordingly it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. The isotopically-labeled compounds can generally be prepared by conventional techniques known to those skilled in the art or by processes analogous to those described preparation of the compound of the invention by using an appropriate isotopically-labeled reagents in place of the non-labeled reagent previously employed.

As used herein, the terms “free form” or “free forms” refers to the compound in non-salt form, such as the base free form or the acid free form of a respective compound, e.g. the compounds specified herein (e.g. mavoglurant or further pharmaceutical active ingredient, for example, as defined herein).

As used herein, the terms “salt”, “salts” or “salt form” refers to an acid addition or base addition salt of a respective compound, e.g. the compounds specified herein (e.g. mavoglurant or further pharmaceutical active ingredient, for example, as defined herein). “Salts” include in particular “pharmaceutically acceptable salts”. The term “pharmaceutically acceptable salts” refers to salts that retain the biological effectiveness and properties of the compounds and, which typically are not biologically or otherwise undesirable. The compounds, as specified herein (e.g. mavoglurant or further pharmaceutical active ingredient, for example, as defined herein), may be capable of forming acid and/or base salts by virtue of the presence of amino and/or carboxyl groups or groups similar thereto. The compound of the invention is capable of forming acid addition salts, thus, as used herein, the term pharmaceutically acceptable salt of mavoglurant means a pharmaceutically acceptable acid addition salt of mavoglurant.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Pharmaceutically acceptable salts can be synthesized from a basic or acidic moiety, by conventional chemical methods. Generally, such salts can be prepared by reacting the free acid forms of the compound with a stoichiometric amount of the appropriate base (such as Na, Ca,

Mg, or K hydroxide, carbonate, bicarbonate or the like), or by reacting the free base form of the compound with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 22^{nd} edition, Mack Publishing Company (2013); and in “Handbook of Pharmaceutical Salts: Properties, Selection, and Use” by Stahl and Wermuth (Wiley-VCH, Weinheim, 2011, 2 ^{nd} edition).

The compounds specified herein (e.g. mavoglurant or the further pharmaceutical active ingredient, for example, as defined herein) can be administered by conventional route, in particular orally, such as in the form of tablets, capsules, caplets or particulates, which can be manufactured according to pharmaceutical techniques as known in the art (for example in “Remington Essentials of Pharmaceutics, 2013, 1^{st} Edition, edited by Linda Felton, published by Pharmaceutical Press 2012, ISBN 978 0 85711 105 0; in particular Chapter 30), wherein pharmaceutical excipients are, for example, as described in “Handbook of Pharmaceutical Excipients, 2012, 7^{th} Edition, edited by Raymond C. Rowe, Paul J. Sheskey, Walter G. Cook and Marian E. Fenton, ISBN 978 0 85711 027 5”. In particular, WO2014/199316 describes formulations comprising mavoglurant, in particular modified release formulations thereof, and is incorporated herein by reference, more particularly the Examples, the preferred embodiments and claims therein.

The pharmaceutical composition or combination of the present invention can be in a unit dosage form (e.g. tablet, capsule, caplet or particulate) comprising an amount ranging of from 1 mg to 300 mg, in particular of from 50 mg to 200 mg, such as 50 mg to 100 mg, more particularly 200 mg, of mavoglurant (referring to an amount of the free form of mavoglurant, and if a salt thereof is used the amount will be adapted accordingly; in particular mavoglurant is in the free form). For the above-mentioned uses/treatment methods the appropriate dosage may vary depending upon a variety of factors, such as, for example, the age, weight, sex, the route of administration or salt employed. In patients with, for example, of from 50-70 kg body weight, an indicated daily dosage is, for example, 200 mg/b.i.d (referring to an amount of the free form of mavoglurant, and if a salt thereof is used the amount will be adapted accordingly).

### ABBREVIATIONS

EtG=Ethyl Glucuronide
DSM 5=Diagnostic and Statistical Manual of Mental Disorders, 5th Ed
CUD=cocaine use disorder
BE=benzoylecgonine
PK=Pharmacokinetic
TLFB=Timeline Follow-Back
mg=milligram
bid=b.i.d=twice (two times) a day
ECG=electrocardiogram
SoA=standard of care
C-SSRS=Columbia Suicide Severity Rating Scale
BDI=Beck's Depression Inventory
STAI=State-trait Anxiety Inventory (2 components: State or S-anxiety and Trait or T-anxiety)
CGI=Clinical Global Impression (2 components: CGI-S [rated illness severity], CGI-I [rated change from baseline]) were also assessed.
FDA=Food and Drug Administration
SE=standard error
Θ=estimated proportion of cocaine use days
LLOQ=lower limit of quantification LLOQ
ULOQ=upper limit of quantification ULOQ

The following Examples serve to illustrate the invention without limiting the scope thereof.

### EXAMPLES

### Example 1: Clinical Trial assessing the effect of Mavoglurant in patients with substance

### use disorder, wherein the substance is an stimulant which is cocaine

Trial Design

This was a Phase 2 randomized, patient- and investigator-blinded, placebo-controlled, parallel-group study.

Trial Participants

Patients diagnosed with Cocaine Use Disorder (CUD) according to Diagnostic and Statistical Manual of Mental Disorders, 5th Ed. (DSM 5) were eligible for inclusion if they used cocaine intranasally as the primary route of administration, had recently used cocaine as confirmed by a positive urine screen for one or more benzoylecgonine (BE), were seeking treatment for cocaine dependence and had a desire to reduce or cease cocaine use.

Patients with current diagnosis of moderate or severe substance use disorder for any other stimulant except cocaine or current treatment for substance use disorder were excluded from the study.

Inclusion Criteria

| | |
|---|---|
| 1. | Male and female subjects, 18 to 65 years of age (inclusive) and diagnosed with CUD |
| | according to DSM-5 |
| 2. | Used cocaine through snorting (intranasally) as primary route of administration. |
| 3. | Recent cocaine use confirmed by positive urine screen for 1 or more BE. |
| 4. | Sought treatment for cocaine dependence and had a desire to reduce or cease cocaine use |
| | as per goals assessed at baseline. |
| 5. | Abstinent from cocaine use for at least 3 days preceding first dosing (Day 1) as assessed |
| | by TLFB. The 2 urine drug screen samples at Visit 2 and Visit 3 must have shown a |
| | decrease in BE levels or were both negative. |
| 6. | In good health as determined by medical history and physical examination at screening. |

Exclusion Criteria

| | |
|---|---|
| 1. | History of hypersensitivity to any of the study treatments or excipients or to drugs of similar |
| | chemical classes. |
| 2. | Had current diagnosis of moderate or severe substance use disorder (according to the |
| | DSM-5) for alcohol, cannabis, opioids, or other stimulants, except cocaine. |
| 3. | Met current or lifetime DSM-5 criteria for schizophrenia or any psychotic disorder or organic |
| | mental disorder. |
| 4. | Had current treatment for substance use disorder (e.g., disulfiram, acamprosate, |
| | methylphenidate, modafinil, topiramate, immediate release dexamphetamine, or baclofen). |
| 5. | Required treatment with any psychoactive medications, including any anti-seizure |
| | medications (with an exception of medications used for short-term treatment of insomnia). |
| | SSRIs were allowed if they had an adequate stable dose for at least 1 month prior to study |
| | treatment dosing. |
| 6. | Pregnant or nursing (lactating) women, where pregnancy was defined as the state of a |
| | female after conception and until the termination of gestation, confirmed by a positive hCG |
| | laboratory test. |
| 7. | History of porphyria. |
| 8. | History or presence of malignancy of any organ system, (other than localized basal cell |
| | carcinoma of the skin or in-situ cervical cancer), treated or untreated, within the past 5 years, |
| | regardless of whether there was evidence of local recurrence or metastases. |
| 9. | History of immunodeficiency diseases, including a positive HIV (ELISA and Western blot) |
| | test result. |
| 10. | Chronic infection with HBV or HCV. |
| 11. | Scored “yes” on item 4 or item 5 of the suicidal ideation section of the C-SSRS, if this |
| | ideation occurred in the past 6 months, or “yes” on any item of the suicidal behavior section, |
| | except for the “non-suicidal self-injurious behavior” (item also included in the suicidal |
| | behavior section), if this behavior occurred in the past 2 years. |
| 12. | Subjects with controlled hypertension, i.e., subjects diagnosed for hypertension and taking |
| | anti-hypertensive treatment were excluded from this study. |

Trial Procedure

This study was conducted in patients with CUD and consisted of about a 21-day screening period followed by a 7-day baseline period, a 98-day outpatient treatment period, and an end of study visit evaluation about 14 days after the last study drug administration (Error! Reference source not found. A). Study treatment was taken twice daily (separated by about 12-hour intervals) with food. For patients in the mavoglurant arm [mavoglurant (free form) provided as modified release formulation (e.g. in WO2014/199316)], an uptitration regimen was performed for 14 days (50 mg b.i.d. from Days 1 to 7 and 100 mg b.i.d from Days 8 to 14) and was followed by 200 mg b.i.d for 84 days.

Endpoints

The primary endpoint was the proportion of cocaine use days during the treatment period, assessed by timeline followback [TLFB] cocaine self-report report. The secondary endpoints included a weekly urine analysis for a log-transformed quantitative measure of cocaine metabolite benzoylecgonine (BE), safety assessments (clinical parameters, suicidal ideation, adverse events [AE] and serious adverse events [SAE] at every visit), and pre- and post-dose levels of mavoglurant in the plasma. Exploratory endpoints included Clinical Global Impression (CGI; 2 components: CGI-S [rated illness severity], CGI-I [rated change from baseline]).

Statistical Analysis

For the primary endpoint, a Bayesian analysis was conducted on the proportion of cocaine use days. It was assumed to be a continuous outcome with a normal distribution. The proportion of cocaine use days over the treatment period was analyzed using an analysis of covariance (ANCOVA) model with baseline use as a covariate. The profiles of consumption over time were compared between treatment groups through analyses of longitudinal data (weekly use and monthly use) using linear mixed model for repeated measures (MMRM). The model evaluated the posterior probability that the difference in proportions of cocaine use days between mavoglurant and placebo was <0 and that it was <−10%. A difference of 10% of days was deemed the minimal clinically relevant effect and a difference of 20% would be a very promising effect based on the literature in this indication. The study was deemed to show a positive signal of efficacy if the following two proof of concept (PoC) criteria were met:

There was at least 90% probability that the difference in the proportions of cocaine use days between mavoglurant and placebo was <0

There was at least 50% probability that the difference in the proportions of cocaine use days between mavoglurant and placebo was <−10%

Estimates of the difference between mavoglurant and placebo were provided with 90% Cls and P values. Sensitivity analyses were to be performed using other models if the distribution of the data was not deemed normal.

Data for vital signs, electrocardiogram (ECG) and clinical laboratory evaluations were summarized by treatment and visit/time. Mavoglurant plasma concentrations were recorded at pre-dose and 2 hour (±1 hour) sampling window after the morning dose and summary statistics for concentrations were provided for plasma.

A sample size of 68 patients (31 patients on mavoglurant and 37 patients on placebo) was determined assuming a standard deviation of 32% for the proportion of cocaine use days. Patients who were withdrawn from the study for reasons other than safety may have been replaced if the dropout rate was higher than anticipated (20% higher). The probability of reaching the target efficacy (success) at the end of the study was 10% if mavoglurant was equal to placebo, and it was >85% if the true difference between mavoglurant and placebo was <−20%. For a sample size of 24 patients per group (assuming 20% of patients excluded from the analysis) the probability of success was >80% if the true difference was <−20%.

Participants

There were 71 patients randomized in total; 33 were randomized to mavoglurant and 38 were randomized to placebo (Error! Reference source not found.B). Among the 33 patients randomized to mavoglurant, two patients were not treated. Among the 38 patients randomized to placebo, one was not treated. Thus, 68 patients were treated in total (31 on mavoglurant and 37 on placebo). Overall, there were 17 patients who did not complete the study primarily due to their own decision—3 untreated, 14 due to discontinuation. Most of the patients were white (98.5%), male (82.4%) and aged between 18 and 57 years.

Analysis of the Primary Endpoint

The primary endpoint was the proportion of cocaine use days by TLFB during the treatment period (Day 1 to last day of treatment). For each subject, the proportion of cocaine use days was calculated by dividing the number of days of cocaine use during the treatment period with the treatment period, i.e., 98 days for completers and number of days between Day 1 and day of last dose in case of premature discontinuation of study treatment. Cocaine consumption was recorded daily (Yes/No) using the TLFB during the entire study.

Analysis of secondary endpoints

Efficacy (Pharmacodynamics)

Quantitative Urine Measurements

Two urine samples per week provided a log-transformed quantitative measure of BE (cocaine's metabolite). The proportions of positive urine samples over the full treatment period were compared between the mavoglurant and placebo groups using a 2-sample t-test. The weekly profiles of the log-transformed quantitative urine BE values were analyzed using a mixed model similar to the model used for weekly proportion of use days, except that no baseline covariate was included.

Alcohol Consumption

Alcohol consumption was recorded by the subjects using TLFB. The number of drinks was recorded daily. The proportion of days of alcohol consumption during the total study treatment period were compared between groups using an ANCOVA model with treatment as factor, country and past alcohol consumption as covariate. The past consumption of alcohol was the proportion of alcohol over the 28 days preceding the screening visit, which was assessed retrospectively using the TLFB. Point estimates and the associated 95% Cls for the difference between mavoglurant and placebo were obtained. The same analysis was repeated on the weekly numbers of drinks.

Abstinence

A subject was considered abstinent the last x weeks of treatment if they completed the 14-week treatment period and were abstinent the last x weeks (with x=1, 2, 3. . . . , 14). If a subject did not complete 14 weeks, it was considered as not abstinent.

Safety

Data for vital signs (BP, pulse rate, body temperature), ECGs, and clinical laboratory evaluations were listed by treatment, subject, visit/time; abnormalities were flagged (if ranges were available).

Adverse Events

All information obtained on AEs are displayed by treatment and subject. The number and percentage of subjects with AEs are tabulated by body system and PT with a breakdown by treatment. A subject with multiple AEs within body system was only counted once towards the total of this body system and treatment.

Pharmacokinetics

The PK analysis set was used for this analysis. Conventional PK parameters were not calculated due to the limited sampling schedule. Mavoglurant plasma concentration data is listed by treatment, subject, and visit/sampling time point. Descriptive summary statistics are provided by treatment and visit/sampling time point, including the frequency (n, %) of concentrations below the LLOQ. Plasma concentrations are listed at pre-dose and 2±1-hour sampling window post-morning dose and summary statistics for concentrations are provided for plasma. The data is part of the overall population PK model. Concentrations below the LLOQ were treated as zero in summary statistics. A geometric mean was not reported if the dataset included zero values.

Analysis of Exploratory Endpoints

Hair Analysis

Hair analysis was used as an adjunct to urine samples collection and helps document drug use history. Hair specimens were collected and at baseline and at the end of the study. Patients with hair shorter than 3 cm or bleached and/or dyed hair (proximal 4 cm) were excluded from this assessment. If only the tip of long hairs were bleached, samples were collected.

The quantitative measures were log-transformed and individual ratio between end of study and baseline was calculated and listed by treatment, subject and visit/time. The log-transformed data profiles over time were explored, compared between treatment groups and the relationship between cocaine and alcohol measures in hair, in urine and timeline follow-back was assessed.

Summary statistics were provided by treatment and visit/time. Heatmaps to visualize the trend in the log-transformed quantitative measures were created. To explore the relationship between the log-transformed quantitative measures of the compounds being assayed and the timeline follow-back cocaine and alcohol measures, scatterplots were created for the end of study results. A regression line was fitted to the scatter plots if a linear relationship was observed. The end of study week results was used for the timeline follow-back information.

Handling of LLOQ and ULOQ

To ensure that the hair analysis quantitative parameters only had numerical values, censored values were imputed as follows
Values below the LLOQ were replaced by LLOQ/2.
Values above the ULOQ were replaced by ULOQ.

Imputed values were used for summary statistics, inferential analyses and plots (with a special symbol). Values below LLOQ and values above ULOQ were shown as such in the listings. In the summary (FIG. S2), the frequency (n, %) of values below the LLOQ and above the ULOQ,

respectively, were included. If the proportion of imputed data was more than 20% for any treatment group at any timepoint, a footnote was added to the summary statistics table stating that the proportion of values outside the limits of quantification was more than 20% for some treatment groups at some time points and that in such cases summary statistics were heavily biased. If the proportion of imputed data for a given measure, across all treatment groups and time points, was more than 50%, no summary statistics were provided and the data were only listed.

Abstinence

A subject was considered abstinent the last x weeks of treatment if they completed the 14-week treatment period and were abstinent the last x weeks (with x=1, 2, 3. . . . , 14). If a subject did not complete 14 weeks, it was considered as not abstinent. Binary response variables based on abstinence were calculated.

This analysis was added in order to inform on abstinence rates as this is an important endpoint in Phase III CUD studies. Binary response variables based on abstinence were calculated.

Clinical Outcome Measures

Clinical Outcome Assessments (COAs)

Clinical Outcome Assessments such as Patient-reported Outcome and Observer-reported Outcome were specific methods for assessment and recording detailing when each assessment was to be performed. Safety assessments are specified below.

Patient Reported Outcome included:
1. The TLFB cocaine: is a cocaine assessment method that obtains estimates of daily cocaine intake and has been evaluated with clinical and nonclinical populations. Using a calendar, people provide retrospective estimates of their daily cocaine use over a specified time period that can vary up to 12 months from the interview date. Several memory aids can be used to enhance recall (e.g., calendar; key dates serve as anchors for reporting cocaine use). The Cocaine TLFB has been shown to have good psychometric characteristics with a variety of cocaine use groups, and can generate variables that provide a wide range of information about an individual's cocaine use (e.g., pattern, variability, and magnitude of cocaine use). The method is recommended for use when relatively precise estimates of cocaine use are necessary, especially when a complete use pre-posttreatment). Clinically, the TLFB can be used to provide feedback about one's cocaine consumption in an effort to increase a client's motivation to change.
2. The TLFB alcohol: is a drinking assessment method that obtains estimates of daily drinking and has been evaluated with clinical and nonclinical populations. Using a calendar, people provide retrospective estimates of their daily drinking over a specified time period that can vary up to 12 months from the interview date. Several memory aids can be used to enhance recall (e.g., calendar; key dates serve as anchors for reporting drinking; standard drink conversion). The Alcohol TLFB has been shown to have good psychometric characteristics with a variety of drinker groups, and can generate variables that provide a wide range of information about an individual's drinking (e.g., pattern, variability, and magnitude of drinking). The method is recommended for use when relatively precise estimates of drinking are necessary, especially when a complete picture of drinking days (i.e., high- and low-risk days) is needed (evaluating drinking pre/post-treatment). Clinically, the TLFB can be used to provide feedback about one's drinking in an effort to increase a client's motivation to change. Overall, the Alcohol TLFB method provides a relatively accurate portrayal of drinking, and has both clinical and research utility.
3. The BDI-II: is instrument endorsed by the National Institute for Health and Clinical Excellence for use in primary care in measuring baseline depression severity and responsiveness to treatment.
4. The STAI: is an assessment tool for measuring the presence and severity of current symptoms of anxiety and a generalized propensity to be anxious. There are 2 subscales within this measure. First, the State Anxiety Scale (S-Anxiety) evaluates the current state of anxiety, asking how respondents feel “right now,” using items that measure subjective feelings of apprehension, tension, nervousness, worry, and activation/arousal of the autonomic nervous system. The Trait Anxiety Scale (T-Anxiety) evaluates relatively stable aspects of “anxiety proneness,” including general states of calmness, confidence, and security.
5. The C-SSRS: The Columbia-Suicide Severity Rating Scale (C-SSRS), is a questionnaire that prospectively assesses Suicidal Ideation and Suicidal Behavior. The C-SSRS must be administered at each visit, including unscheduled visits. The C-SSRS, which uses a semi-structured interview to probe subject responses, was administered by an individual who has received training and certification in its administration. At the first study visit, the “baseline/screening” version of the C-SSRS, was administered. This version assesses Suicidal Ideation and Suicidal Behavior during the subject's lifetime and during a predefined period. At subsequent visits, the “since last visit” version was administered. If, at any time after screening and/or baseline, the score is “yes” on item 4 or item 5 of the Suicidal Ideation section of the C-SSRS or “yes” on any item of the Suicidal Behavior section, the subject must be referred to a mental health care professional for further assessment and/or treatment. The decision on whether the study treatment should be discontinued was to be taken by the investigator in consultation with the mental health professional to whom the subject is referred. In addition, all life-threatening events must be reported as SAEs. For example, if a subject answers “yes” to one of the questions in the Suicidal Behavior section, an SAE must be reported if the event was life-threatening. All events of “Non-Suicidal Self-Injurious Behavior” (question also included in the Suicidal Behavior section) were reported as AEs and assigned the appropriate severity grade.

Observer Reported Outcome:
1. Clinical Global Impression (CGI) is a rating scale which measures the severity of symptoms, treatment response and the efficacy of treatments in treatment studies of patients with mental disorders. CGI provides an overall clinician-determined summary measure that takes into account all available information, including a knowledge of the patient's history, psychosocial circumstances, symptoms, behavior, and the impact of the symptoms on the patient's ability to function. CGI is rated by an experienced clinician who is familiar with the disease under study and the likely progression of treatment. The CGI is rated without regard to the clinician's belief that any clinical changes are or are not due to medication and without consideration of the etiology of the symptoms. As noted above, the CGI has two components: the CGI-Severity, which rates illness severity, and the CGI-Improvement, which rates change from the initiation (baseline) of treatment.

Handling of missing values/censoring/discontinuations

In the case of premature discontinuation of study treatment, the calculation of the primary variable was done on the period with both TLFB data available and when study treatment was taken. No missing data was expected between visits per the TLFB completion guidelines and investigator's checks at the visits. For sensitivity analyses, the incomplete profiles were included in longitudinal analyses

### Results

Primary endpoint

As assessed by cocaine TLFB, following the 98-day administration of mavoglurant/placebo, the posterior probability in reducing cocaine use was 99.0% for treatments difference <0, and 36.6% for treatments difference <−10%. The difference between groups (mavoglurant-placebo) was statistically significant (P=0.021) (Error! Reference source not found. Error! Reference source not found. A and Error! Reference source not found. A).
a) Primary analysis (Error! Reference source not found. B)
PoC criteria
   1. Bayesian analysis over the treatment period (14 weeks) P(θ_{mavoglurant}−θ_{placebo}<0 data)=0.999 [PoC criterion 1 is met (P>0.90)]
   2. P(θ_{mavoglurant}−θ_{placebo}<−10% |data)=0.366 [PoC criterion 2 is not met (P<0.50, and the difference between groups is 8.7%<10%)]
b) Supplementary analyses: MMRM showed a difference between groups in the monthly proportions of cocaine use. Monthly proportions were statistically different at Months 2, 3 5 and 4. At Month 2, a difference of 8% was reached (P=0.042), while at Month 3, a difference of 14% was reached (P=0.003) (Error! Reference source not found. C).

Secondary and exploratory endpoints Mavoglurant significantly reduced cocaine consumption, as assessed by urine cocaine (BE), in comparison to placebo (P=0.025) (Error! Reference source not found.).

Mavoglurant was associated with significantly enhanced clinical improvement when compared to placebo, as assessed by CGI (‘very much’ and ‘much’ improved: 90.9% for mavoglurant vs 46.6% for placebo) (Error! Reference source not found. 3C). Improvement from baseline (CGI-I) was highly significant for mavoglurant as compared to placebo (P<0.001).

Based on the amount of money spent on cocaine from baseline, a greater proportion of patients in the mavoglurant group (41.4%) vs placebo group (16.7%) showed cocaine abstinence in the last 3 weeks of the study (P=0.040), as defined by the self-report (TLFB) and a negative urine BE (FIG. S1). Similar results were observed for alcohol abstinence (FIG. 4). Mavoglurant reduced anxiety and depressive symptoms, as assessed by the STAI and BDI, but the difference vs placebo was not statistically significant (P=0.144 for S-Anxiety and P=0.425 for T-Anxiety; P=0.696 for BDI-II) (Error! Reference source not found. 3A; FIG. S3B).

In the hair analysis, cocaine and its metabolite amphetamine showed higher reductions from baseline as compared to placebo (FIG. S2).

Mavoglurant reduced alcohol consumption, as assessed by TLFB, although the difference vs placebo was not statistically significant (P=0.072) (FIG. 6; FIG. 7).

Safety and Adverse Effects

No deaths were reported during the study (FIG. 5), and there were no significant findings in vital signs, ECGs or clinical safety laboratory parameters. A minimal number of patients had active suicidal ideation, as assessed by the Columbia-Suicide Severity Rating Scale (C-SSRS).

The AEs with higher incidences were related to the central nervous system (FIG. S3). The overall AE rate was comparable between the mavoglurant group and the placebo group, but the mavoglurant group had a higher rate of psychiatric symptoms. Drug-related AEs were observed to disappear after several days. Pharmacokinetic results are reported in FIG. S3D.

Summary of Results

The posterior probability of mavoglurant reducing cocaine use at end of treatment (EOT) was 99.0% for treatments difference <0, and 36.6% for treatments difference <−10%. The difference between mavoglurant and placebo was statistically significant (P=0.021). At EOT, there were significantly lower levels of BE in urine vs placebo (P=0.025). Depressive and anxiety symptoms were reduced nonsignificantly and global functioning was significantly improved in 90.9% patients in the treated group vs placebo (46.6%) (P<0.001). Mavoglurant was safe and well-tolerated and 79% of the patients completed the study at EOT.

Our data (see Figures, as referred above and depicted below) shows that mavoglurant is safe and well tolerated in CUD patients, with good tolerability, no withdrawal and no deaths or SAEs in the mavoglurant group. Mavoglurant reduced cocaine consumption significantly in CUD patients. The PoC criterion 2 was not met, but it is important to note that the reason for this was that the effect of mavoglurant on reducing cocaine use increased over time and was most distinct at the end of the study. The predefined statistical analysis included the time that the patients were on mavoglurant and not just the last month when the effect was most prominent. Compared to those in the placebo group (16.7%), a greater proportion of patients in the mavoglurant group (41.4%) abstained from cocaine use in the last 3 weeks of the study (P=0.040), as defined by the self-report (TLFB) and a negative urine BE. There were lower levels of BE in urine after the 98-day administration of mavoglurant in comparison with placebo, which confirmed the results of the assessment by TLFB. The improvement of global functioning as measured by Clinical Global Impression—Improvement scale was consistent with the reduced use of cocaine associated with the administration of mavoglurant. Global functioning for the mavoglurant group was more improved than the placebo group at Day 98, which aligned with cocaine reduction. In the mavoglurant group, 90.9% patients were ‘very much’ or ‘much’ improved at Day 98 vs 46.6% on placebo.

### Example 2: Clinical Trial assessing the effect of Mavoalurant in patients with substance use disorder, wherein the substance is an stimulant which is an amphetamine-type stimulant (e.g. methamphetamine)

Study Objectives and Endpoints

| Primary objective(s) | Endpoints related to primary objective(s) |
|---|---|
| To evaluate treatment effect of 98-day | Proportion of amphetamine-type |
| mavoglurant administration in reducing | stimulant (e.g. methamphetamine) use |
| amphetamine-type stimulant (e.g. | days by TLFB amphetamine-type |
| methamphetamine) use | stimulant (e.g. methamphetamine) self- |
| | report |
| Secondary objective(s) | Endpoints related to secondary objective(s) |
| To assess the effects of 98-day | Urinalysis [e.g. by measuring |
| mavoglurant administration versus | metabolites of an amphetamine-type |
| placebo on: | stimulant in urine, such as metabolites |
| a) other measures of amphetamine-type | of methamphetamine] |
| stimulant (e.g. methamphetamine) use | b)TLFB alcohol self-report; urinalysis [Ethyl |
| b) alcohol use | Glucuronide (EtG)] |
| To assess the safety and tolerability of | Vital signs, ECG parameters, clinical safety |
| multiple bid oral doses of mavoglurant | laboratory parameters |
| | (chemistry/hematology/urinalysis), |
| | (serious) adverse events reporting, suicidal |
| | ideation [Columbia Suicide Severity Rating |
| | Scale (C-SSRS)] |
| To evaluate the pharmacokinetics of | mavoglurant plasma concentrations (pre- |
| mavoglurant | and post-dose levels) |
| Exploratory objective(s) | Endpoints related to exploratory objective(s) |
| To examine whether individual genetic | genetic markers in pharmacogenetic or |
| variation in genes relating to drug | biologic candidate genes for substance use |
| metabolism and transporters, substance | disorder, wherein the substance is |
| use disorder, wherein the substance is | amphetamine-type stimulant (e.g. |
| amphetamine-type stimulant (e.g. | methamphetamine), target or drug |
| methamphetamine), and the drug target | response |
| pathway confer differential response to | |
| mavoglurant | |
| To assess the effects of 98-day | a) Beck's Depression Inventory (BDI) |
| mavoglurant administration versus | b) State-Trait Anxiety Inventory (STAI) |
| placebo on: | c) Clinical Global Impression Scale - Severity |
| a) depressive symptoms | (CGI-S) and Improvement (CGI-I) |
| b) anxiety symptoms | |
| c) global functioning | |

Study Design

This is a randomized, subject and investigator blinded, parallel group, placebo-controlled study in patients with substance use disorder, wherein the substance is amphetamine-type stimulant (e.g. methamphetamine). The study consists of: about 21-day screening period followed by a 7-day baseline; a 98-day out-patient treatment period (14-day up-titration dose regimen followed by 84-day maintenance dose) and finally an end of study visit evaluation approximately 14-days after the last study drug administration. The total duration for each patient in the study is up to approximately 20-weeks.

Study visits (days 1-112): study visits are performed in an ambulatory setting of frequency twice per week. During these visits the urine samples are collected for drug screen (and others drug metabolites) along with safety/efficacy assessments.

Screening (days −28 to −8): includes safety examinations and other clinical tests, determines patients' initial eligibility. Patients who meet the eligibility criteria at screening are admitted to the baseline evaluation.

Baseline (days −7 to −1): includes, in addition to the safety evaluations, a patient's self-assessment on various scales and questionnaires. During baseline, a history of self-reported amphetamine-type stimulant (e.g. methamphetamine) use (TLFB) and two urine samples are collected on two different days with second sample collected 3 days prior randomization to demonstrate the abstinence from amphetamine-type stimulant (e.g. methamphetamine) use. Treatment (days 1-98): following baseline, on Day 1, eligible patients are randomly assigned in a 1:1 ratio to either mavoglurant (free form) or placebo
Group A—mavoglurant (free form): up-titration regimen for the first 2 weeks: 50 mg bid from Day 1 to Day 7,100 mg bid from Day 8 to Day 14, followed by dosing at 200 mg bid for 84-days The dose selected (200 mg b.i.d/modified release formulation) for evaluation in this study has been chosen based on the safety, tolerability and pharmacokinetic data from completed mavoglurant studies.
Group B: matching placebo.

Study drug: mavoglurant (free form) provided in modified release formulation (e.g. in WO2014/199316) and taken twice daily (b.i.d.), in the morning and evening (separated by approximately 12 hour intervals), with food. For all ambulatory morning visits that involve any study assessments or PK/urine sample collection, study medication is self-administered by patient at study center and supervised by study personnel. On these days, standard breakfast is served at study center and consumed by the patient during his/her medication intake.

During treatment period, patients also undergo assessments with various scales and questionnaires, as well as safety assessments and pharmacokinetic sampling at pre- and 2±1-hour post dose per SoA.

Urine samples (days 1-113): samples are collected at study center twice per week, with at least 48 hours separating tests, e.g. Tuesday and Fridays or Mondays and Thursdays or Tuesday morning and Thursday afternoon. The sample collection is staff-observed and is assayed quantitatively for the presence of amphetamine-type stimulant (e.g. methamphetamine) metabolites. Urine samples are collected during study conduct: 28 samples from patients who remain in treatment for the 14 weeks (4 samples in weeks 1-2 of up-titration); 24 samples in weeks 3-14 (maintenance dose); 4 samples in weeks 15-16 (follow up) and finally last 1 sample at end of study visit. If a patient fails to attend the clinic or refuses to provide a sample on a scheduled testing day, samples are considered positive unless an excused absence is granted (e.g. illness, other personal reason). In cases of missed or refused samples, samples are collected on the next day whenever possible.

Clinical support to ensure medication and protocol's adherence:
Medication compliance: patients are at the study site at time of study drug administration for the morning dose on PK collection days and on all other days that involves urine sampling assessments. On these days study medication self-administration is supervised by study personnel, compliance is ensured by mouth check after the medication is swallowed. To monitor medication adherence, patients are provided with individual Medication Diary (booklet) to record administration of study medication. Medication compliance is monitored by the investigator and/or study personnel at least on a weekly basis using tablet(s) counts. Dosage adherence is verified by comparing the patient's Medication Diary self-reported data against the total number of tablets in the returned bottle or blister (depends on the packaging form). Adherence is calculated as the total amount of tablets taken divided by the scheduled total amount to be taken during the treatment phase. If the Investigator feels it is appropriate, the patient may also be contacted during the out-patient periods to confirm compliance.

Population

Patients, aged of 18-65 inclusive with a diagnosis of substance use disorder, wherein the substance is amphetamine-type stimulant (e.g. methamphetamine), are enrolled in the study.

Statistical model and method of analysis

The primary variable: is the proportion of amphetamine-type stimulant (e.g. methamphetamine) use days during the treatment period (days 1-98). For each patient, the proportion of amphetamine-type stimulant (e.g. methamphetamine) use days is calculated by dividing the number of days of amphetamine-type stimulant (e.g. methamphetamine) use during the treatment period, i.e. 98 days for completers and number of days between Day 1 and day of last dose in case of premature discontinuation of study treatment. It is considered as a continuous variable. The amphetamine-type stimulant (e.g. methamphetamine) consumption is recorded daily (Yes/No) using the TLFB during the entire study.

A Bayesian analysis is conducted on the proportion of amphetamine-type stimulant (e.g. methamphetamine) use days. It is assumed to be a continuous outcome with normally distributed errors. A linear model with treatment factor and past consumption of amphetamine-type stimulant (e.g. methamphetamine) as covariate is fitted. Non informative priors are used for all parameters. The past consumption of amphetamine-type stimulant (e.g. methamphetamine) is the proportion of amphetamine-type stimulant (e.g. methamphetamine) use days over the 3 months preceding the screening visit, which is assessed using the TLFB.

Non informative priors are used for all parameters. The past consumption of amphetamine-type stimulant (e.g. methamphetamine) is the proportion of amphetamine-type stimulant (e.g. methamphetamine) use days over the 3 months preceding the screening visit, which is assessed using the TLFB. The model evaluates the posterior probability that the difference in the proportions of amphetamine-type stimulant (e.g. methamphetamine) use days between mavoglurant and placebo is <0 and that it's<−10%. The difference of 10% of days is deemed the minimal clinically relevant effect and a difference of 20% is a very promising effect, based on the literature in this indication. The study shows a positive signal of efficacy if the 2 following criteria are met:
1. there is at least 90% probability that the difference in the proportions of amphetamine-type stimulant (e.g. methamphetamine) use days between mavoglurant and placebo is <0
2. there is at least 50% probability that the difference in the proportions of amphetamine-type stimulant (e.g. methamphetamine) use days between mavoglurant and placebo is <−10% Sensitivity analyses are performed using other models, like negative binomial regression if the distribution of the data is not normal. Additionally, the profiles of consumption over time are compared between treatment groups through analyses of longitudinal data (weekly use) using mixed models for repeated measures.

The secondary variables: include the proportion of negative UDS over the treatment period and will be analyzed in the same way as the primary variable. Safety and PK are secondary endpoints for this study.

1. A method for treating substance use disorder, wherein the substance is an amphetamine-type stimulant, in a patient in need thereof, comprising administering to said patient an effective amount of mavoglurant, or a pharmaceutically acceptable salt thereof.

2. A method for the reduction of substance use by a substance use disorder patient, in need thereof, wherein the substance is an amphetamine-type stimulant, comprising administering to said patient an effective amount of mavoglurant, or a pharmaceutically acceptable salt thereof.

3. A method for preventing relapse into substance use by a substance use disorder patient, in need thereof, wherein the substance is an amphetamine-type stimulant, comprising administering to said patient an effective amount of mavoglurant, or a pharmaceutically acceptable salt thereof.

4. A method for the promotion of substance abstinence by a substance use disorder patient, in need thereof, wherein the substance is an amphetamine-type stimulant, comprising administering to said patient an effective amount of mavoglurant, or a pharmaceutically acceptable salt thereof.

5. A method for treating the symptoms of depression or anxiety associated with substance use disorder, wherein the substance is an amphetamine-type stimulant, comprising administering to said patient an effective amount of mavoglurant, or a pharmaceutically acceptable salt thereof.

6. The method according to anyone of claim 1, wherein the amphetamine-type stimulant use is inhalation, intravenous injection, nasal insufflation or oral ingestion of the amphetamine-type stimulant.

7. The method according to claim 1, wherein the substance use disorder is comorbid with a psychiatric disorder, such as antisocial personality disorder, borderline personality disorder, depression, anxiety, schizophrenia, attention deficit hyperactivity disorder, bipolar disorder, obsessive compulsive disorder or binge eating disorder.

8. The method according to claim 1, wherein the method is combined with standardized psychological treatment, for example, at individual or group level.

9. The method according to claim 1, wherein the method is combined with psychosocial or behavioral therapy or combination thereof, in particular contingency management based therapy.

10. The method according to claim 9, wherein the psychosocial or the behavioral therapy is computer-assisted.

11. The method according to claim 1, wherein mavoglurant, or a pharmaceutically acceptable salt thereof, is administered in combination with suvorexant, bupropion or mirtazapine, or salts thereof, in particular mirtazapine or a salt thereof.

12. The method according to claim 1, wherein mavoglurant, or a pharmaceutically acceptable salt thereof, is administered in combination with a further active agent, for example wherein the further active agent is selected from the group consisting of an antidepressant, an antipsychotic and an anxiolytic.

13. The method according to claim 1, wherein the patient has a genetic variation associated with a substance use disorder.

14. The method according to claim 1, wherein mavoglurant, or a pharmaceutically acceptable salt thereof, is administered in an immediate-release form or a modified-release form.

15. The method according to claim 1, wherein mavoglurant, or a pharmaceutically acceptable salt thereof, is administered in an amount of from 50 mg/b.i.d to 200 mg/b.i.d, in particular 50 mg/b.i.d., 100 mg/b.i.d or 200 mg/b.i.d., such as 200 mg/b.i.d.

16. The method according to claim 1, wherein the substance use disorder is associated with binge drinking or alcohol use disorder.

17. The method according to claim 1, wherein the amphetamine-type stimulant is selected from the group consisting of amphetamine, dextroamphetamine, methamphetamine, methylphenidate, methcathinone, fenetylline, ephedrine, pseudoephedrine, methylphenidate, 3,4-methylenedioxymethamphetamine, diethylpropion, lisdexamfetamine, phentermine, benzphetamine, mephentermine, midoamfetamine, tenamphetamine, 4-hydroxyamphetamine and mephedrone.

18. The method according to claim 1, wherein the amphetamine-type stimulant is methamphetamine.

19. The method according to claim 1, wherein the substance use disorder is mild substance use disorder, moderate substance use disorder or severe substance use disorder.
